SEQUENCE LISTING (211 sequences)

SEQ ID NO: 1 - protein, 6 aa, Artificial Sequence
  peptide
EEIVPN

SEQ ID NO: 2 - protein, 7 aa, Artificial Sequence
  peptide
SEEIVPN

SEQ ID NO: 3 - protein, 8 aa, Artificial Sequence
  peptide
SSEEIVPN

SEQ ID NO: 4 - protein, 6 aa, Artificial Sequence
  peptide
LTDVEN

SEQ ID NO: 5 - protein, 13 aa, Artificial Sequence
  peptide
SEEQQQTEDELQD

SEQ ID NO: 6 - protein, 6 aa, Artificial Sequence
  peptide
HNSLPQ

SEQ ID NO: 7 - protein, 12 aa, Artificial Sequence
  peptide
LPVPQKAVPYPQ

SEQ ID NO: 8 - protein, 13 aa, Artificial Sequence
  peptide
VLPVPQKAVPYPQ

SEQ ID NO: 9 - protein, 7 aa, Artificial Sequence
  peptide
DMPIQAF

SEQ ID NO: 10 - protein, 10 aa, Artificial Sequence
  peptide
DELQDKIHPF

SEQ ID NO: 11 - protein, 11 aa, Artificial Sequence
  peptide
EDELQDKIHPF

SEQ ID NO: 12 - protein, 17 aa, Artificial Sequence
  peptide
EEQQQTEDELQDKIHPF

SEQ ID NO: 13 - protein, 16 aa, Artificial Sequence
  peptide
EQQQTEDELQDKIHPF

SEQ ID NO: 14 - protein, 7 aa, Artificial Sequence
  peptide
QDKIHPF

SEQ ID NO: 15 - protein, 15 aa, Artificial Sequence
  peptide
QQQTEDELQDKIHPF

SEQ ID NO: 16 - protein, 14 aa, Artificial Sequence
  peptide
QQTEDELQDKIHPF

SEQ ID NO: 17 - protein, 13 aa, Artificial Sequence
  peptide
QTEDELQDKIHPF

SEQ ID NO: 18 - protein, 18 aa, Artificial Sequence
  peptide
SEEQQQTEDELQDKIHPF

SEQ ID NO: 19 - protein, 12 aa, Artificial Sequence
  peptide
TEDELQDKIHPF

SEQ ID NO: 20 - protein, 16 aa, Artificial Sequence
  peptide
DIGSESTEDQAMEDIK

SEQ ID NO: 21 - protein, 18 aa, Artificial Sequence
  peptide
DIGSESTEDQAMEDIKQM

SEQ ID NO: 22 - protein, 14 aa, Artificial Sequence
  peptide
ESTEDQAMEDIKQM

SEQ ID NO: 23 - protein, 17 aa, Artificial Sequence
  peptide
ESTEDQAMEDIKQMEAE

SEQ ID NO: 24 - protein, 7 aa, Artificial Sequence
  peptide
FQSEEQQ

SEQ ID NO: 25 - protein, 16 aa, Artificial Sequence
  peptide
GSESTEDQAMEDIKQM

SEQ ID NO: 26 - protein, 17 aa, Artificial Sequence
  peptide
GSESTEDQAMEDIKQME

SEQ ID NO: 27 - protein, 6 aa, Artificial Sequence
  peptide
HNSLPQ

SEQ ID NO: 28 - protein, 18 aa, Artificial Sequence
  peptide
IGSESTEDQAMEDIKQME

SEQ ID NO: 29 - protein, 17 aa, Artificial Sequence
  peptide
IGSESTEDQAMEDIKQM

SEQ ID NO: 30 - protein, 19 aa, Artificial Sequence
  peptide
KDIGSESTEDQAMEDIKQM

SEQ ID NO: 31 - protein, 18 aa, Artificial Sequence
  peptide
SEEQQQTEDELQDKIHPF

SEQ ID NO: 32 - protein, 13 aa, Artificial Sequence
  peptide
SESTEDQAMEDIK

SEQ ID NO: 33 - protein, 15 aa, Artificial Sequence
  peptide
SESTEDQAMEDIKQM

SEQ ID NO: 34 - protein, 16 aa, Artificial Sequence
  peptide
SESTEDQAMEDIKQME

SEQ ID NO: 35 - protein, 18 aa, Artificial Sequence
  peptide
SESTEDQAMEDIKQMEAE

SEQ ID NO: 36 - protein, 8 aa, Artificial Sequence
  peptide
SSEEIVPN

SEQ ID NO: 37 - protein, 13 aa, Artificial Sequence
  peptide
STEDQAMEDIKQM

SEQ ID NO: 38 - protein, 14 aa, Artificial Sequence
  peptide
STEDQAMEDIKQME

SEQ ID NO: 39 - protein, 16 aa, Artificial Sequence
  peptide
STEDQAMEDIKQMEAE

SEQ ID NO: 40 - protein, 13 aa, Artificial Sequence
  peptide
SESTEDQAMEDIK

SEQ ID NO: 41 - protein, 15 aa, Artificial Sequence
  peptide
SESTEDQAMEDIKQM

SEQ ID NO: 42 - protein, 16 aa, Artificial Sequence
  peptide
SESTEDQAMEDIKQME

SEQ ID NO: 43 - protein, 18 aa, Artificial Sequence
  peptide
SESTEDQAMEDIKQMEAE

SEQ ID NO: 44 - protein, 17 aa, Artificial Sequence
  peptide
SLVYPFPGPIHNSLPQN

SEQ ID NO: 45 - protein, 17 aa, Artificial Sequence
  peptide
SLVYPFPGPIPNSLPQN

SEQ ID NO: 46 - protein, 6 aa, Artificial Sequence
  peptide
LEIVPN

SEQ ID NO: 47 - protein, 12 aa, Artificial Sequence
  peptide
KYKVPQLEIVPN

SEQ ID NO: 48 - protein, 12 aa, Artificial Sequence
  peptide
SLVYPFPGPIPN

SEQ ID NO: 49 - protein, 13 aa, Artificial Sequence
  peptide
FPKYPVEPFTESQ

SEQ ID NO: 50 - protein, 12 aa, Artificial Sequence
  peptide
PKYPVEPFTESQ

SEQ ID NO: 51 - protein, 15 aa, Artificial Sequence
  peptide
MPFPKYPVEPFTESQ

SEQ ID NO: 52 - protein, 18 aa, Artificial Sequence
  peptide
HKEMPFPKYPVEPFTESQ

SEQ ID NO: 53 - protein, 16 aa, Artificial Sequence
  peptide
EMPFPKYPVEPFTESQ

SEQ ID NO: 54 - protein, 7 aa, Artificial Sequence
  peptide
TLTDVEN

SEQ ID NO: 55 - protein, 10 aa, Artificial Sequence
  peptide
VYPFPGPIHN

SEQ ID NO: 56 - protein, 12 aa, Artificial Sequence
  peptide
SLVYPFPGPIHN

SEQ ID NO: 57 - protein, 11 aa, Artificial Sequence
  peptide
LVYPFPGPIHN

SEQ ID NO: 58 - protein, 25 aa, Artificial Sequence
  peptide
TPVVVPPFLQPEVMGVSKVKEAMAP

SEQ ID NO: 59 - protein, 6 aa, Artificial Sequence
  peptide
LPPITQ

SEQ ID NO: 60 - protein, 5 aa, Artificial Sequence
  peptide
PPITQ

SEQ ID NO: 61 - protein, 7 aa, Artificial Sequence
  peptide
NLPPITQ

SEQ ID NO: 62 - protein, 14 aa, Artificial Sequence
  peptide
VYPFPGPIHNSLPQ

SEQ ID NO: 63 - protein, 7 aa, Artificial Sequence
  peptide
KAVPYPQ

SEQ ID NO: 64 - protein, 19 aa, Artificial Sequence
  peptide
SLSQSKVLPVPQKAVPYPQ

SEQ ID NO: 65 - protein, 13 aa, Artificial Sequence
  peptide
YPFPGPIHNSLPQ

SEQ ID NO: 66 - protein, 15 aa, Artificial Sequence
  peptide
LVYPFPGPIHNSLPQ

SEQ ID NO: 67 - protein, 16 aa, Artificial Sequence
  peptide
SLVYPFPGPIHNSLPQ

SEQ ID NO: 68 - protein, 8 aa, Artificial Sequence
  peptide
TLTDVENL

SEQ ID NO: 69 - protein, 12 aa, Artificial Sequence
  peptide
TEDELQDKIHPF

SEQ ID NO: 70 - protein, 6 aa, Artificial Sequence
  peptide
SLVYPF

SEQ ID NO: 71 - protein, 9 aa, Artificial Sequence
  peptide
ELQDKIHPF

SEQ ID NO: 72 - protein, 15 aa, Artificial Sequence
  peptide
NIPPLTQTPVVVPPF

SEQ ID NO: 73 - protein, 14 aa, Artificial Sequence
  peptide
HKEMPFPKYPVEPF

SEQ ID NO: 74 - protein, 10 aa, Artificial Sequence
  peptide
DELQDKIHPF

SEQ ID NO: 75 - protein, 7 aa, Artificial Sequence
  peptide
ASPEVIE

SEQ ID NO: 76 - protein, 8 aa, Artificial Sequence
  peptide
EASPEVIE

SEQ ID NO: 77 - protein, 17 aa, Artificial Sequence
  peptide
EASPEVIESPPEINTVQ

SEQ ID NO: 78 - protein, 13 aa, Artificial Sequence
  peptide
KDIGSESTEDQAM

SEQ ID NO: 79 - protein, 15 aa, Artificial Sequence
  peptide
KFQSEEQQQTEDELQ

SEQ ID NO: 80 - protein, 7 aa, Artificial Sequence
  peptide
MESTEVF

SEQ ID NO: 81 - protein, 12 aa, Artificial Sequence
  peptide
SEEQQQTEDELQ

SEQ ID NO: 82 - protein, 18 aa, Artificial Sequence
  peptide
SEEQQQTEDELQDKIHPF

SEQ ID NO: 83 - protein, 7 aa, Artificial Sequence
  peptide
TLEASPE

SEQ ID NO: 84 - protein, 19 aa, Artificial Sequence
  peptide
TLEASPEVIESPPEINTVQ

SEQ ID NO: 85 - protein, 19 aa, Artificial Sequence
  peptide
TLEDSPEVIESPPEINTVQ

SEQ ID NO: 86 - protein, 16 aa, Artificial Sequence
  peptide
VPQLEIVPNSAEERLH

SEQ ID NO: 87 - protein, 17 aa, Artificial Sequence
  peptide
SLVYPFPGPIPNSLPQN

SEQ ID NO: 88 - protein, 20 aa, Artificial Sequence
  peptide
SLTLTDVENLHLPLPLLQSW

SEQ ID NO: 89 - protein, 15 aa, Artificial Sequence
  peptide
DVENLHLPLPLLQSW

SEQ ID NO: 90 - protein, 14 aa, Artificial Sequence
  peptide
VENLHLPLPLLQSW

SEQ ID NO: 91 - protein, 12 aa, Artificial Sequence
  peptide
NLHLPLPLLQSW

SEQ ID NO: 92 - protein, 11 aa, Artificial Sequence
  peptide
LHLPLPLLQSW

SEQ ID NO: 93 - protein, 10 aa, Artificial Sequence
  peptide
HLPLPLLQSW

SEQ ID NO: 94 - protein, 16 aa, Artificial Sequence
  peptide
AQTQSLVYPFPGPIPN

SEQ ID NO: 95 - protein, 12 aa, Artificial Sequence
  peptide
SLVYPFPGPIPN

SEQ ID NO: 96 - protein, 32 aa, Artificial Sequence
  peptide
VMGVSKVKEAMAPKHKEMPFPKYPVEPFTESQ

SEQ ID NO: 97 - protein, 30 aa, Artificial Sequence
  peptide
GVSKVKEAMAPKHKEMPFPKYPVEPFTESQ

SEQ ID NO: 98 - protein, 27 aa, Artificial Sequence
  peptide
KVKEAMAPKHKEMPFPKYPVEPFTESQ

SEQ ID NO: 99 - protein, 24 aa, Artificial Sequence
  peptide
EAMAPKHKEMPFPKYPVEPFTESQ

SEQ ID NO: 100 - protein, 23 aa, Artificial Sequence
  peptide
AMAPKHKEMPFPKYPVEPFTESQ

SEQ ID NO: 101 - protein, 22 aa, Artificial Sequence
  peptide
MAPKHKEMPFPKYPVEPFTESQ

SEQ ID NO: 102 - protein, 21 aa, Artificial Sequence
  peptide
APKHKEMPFPKYPVEPFTESQ

SEQ ID NO: 103 - protein, 19 aa, Artificial Sequence
  peptide
KHKEMPFPKYPVEPFTESQ

SEQ ID NO: 104 - protein, 18 aa, Artificial Sequence
  peptide
HKEMPFPKYPVEPFTESQ

SEQ ID NO: 105 - protein, 17 aa, Artificial Sequence
  peptide
KEMPFPKYPVEPFTESQ

SEQ ID NO: 106 - protein, 15 aa, Artificial Sequence
  peptide
MPFPKYPVEPFTESQ

SEQ ID NO: 107 - protein, 14 aa, Artificial Sequence
  peptide
PFPKYPVEPFTESQ

SEQ ID NO: 108 - protein, 12 aa, Artificial Sequence
  peptide
PKYPVEPFTESQ

SEQ ID NO: 109 - protein, 6 aa, Artificial Sequence
  peptide
PFTESQ

SEQ ID NO: 110 - protein, 7 aa, Artificial Sequence
  peptide
SVLSLSQ

SEQ ID NO: 111 - protein, 7 aa, Artificial Sequence
  peptide
TLTDVEN

SEQ ID NO: 112 - protein, 8 aa, Artificial Sequence
  peptide
QTEDELQD

SEQ ID NO: 113 - protein, 7 aa, Artificial Sequence
  peptide
TEDELQD

SEQ ID NO: 114 - protein, 14 aa, Artificial Sequence
  peptide
NIPPLTQTPVVVPP

SEQ ID NO: 115 - protein, 13 aa, Artificial Sequence
  peptide
DELQDKIHPFAQT

SEQ ID NO: 116 - protein, 17 aa, Artificial Sequence
  peptide
QTEDELQDKIHPFAQTQ

SEQ ID NO: 117 - protein, 14 aa, Artificial Sequence
  peptide
DELQDKIHPFAQTQ

SEQ ID NO: 118 - protein, 13 aa, Artificial Sequence
  peptide
ELQDKIHPFAQTQ

SEQ ID NO: 119 - protein, 11 aa, Artificial Sequence
  peptide
QDKIHPFAQTQ

SEQ ID NO: 120 - protein, 10 aa, Artificial Sequence
  peptide
DKIHPFAQTQ

SEQ ID NO: 121 - protein, 29 aa, Artificial Sequence
  peptide
NLHLPLPLLQSWMHQPHQPLPPTVMFPPQ

SEQ ID NO: 122 - protein, 22 aa, Artificial Sequence
  peptide
LLQSWMHQPHQPLPPTVMFPPQ

SEQ ID NO: 123 - protein, 21 aa, Artificial Sequence
  peptide
LQSWMHQPHQPLPPTVMFPPQ

SEQ ID NO: 124 - protein, 19 aa, Artificial Sequence
  peptide
SWMHQPHQPLPPTVMFPPQ

SEQ ID NO: 125 - protein, 18 aa, Artificial Sequence
  peptide
WMHQPHQPLPPTVMFPPQ

SEQ ID NO: 126 - protein, 17 aa, Artificial Sequence
  peptide
MHQPHQPLPPTVMFPPQ

SEQ ID NO: 127 - protein, 16 aa, Artificial Sequence
  peptide
HQPHQPLPPTVMFPPQ

SEQ ID NO: 128 - protein, 15 aa, Artificial Sequence
  peptide
SVLSLSQSKVLPVPQ

SEQ ID NO: 129 - protein, 22 aa, Artificial Sequence
  peptide
SVLSLSQSKVLPVPQKAVPYPQ

SEQ ID NO: 130 - protein, 19 aa, Artificial Sequence
  peptide
SLSQSKVLPVPQKAVPYPQ

SEQ ID NO: 131 - protein, 12 aa, Artificial Sequence
  peptide
SLSQSKVLPVPQ

SEQ ID NO: 132 - protein, 18 aa, Artificial Sequence
  peptide
LSQSKVLPVPQKAVPYPQ

SEQ ID NO: 133 - protein, 11 aa, Artificial Sequence
  peptide
LSQSKVLPVPQ

SEQ ID NO: 134 - protein, 17 aa, Artificial Sequence
  peptide
SQSKVLPVPQKAVPYPQ

SEQ ID NO: 135 - protein, 10 aa, Artificial Sequence
  peptide
SQSKVLPVPQ

SEQ ID NO: 136 - protein, 15 aa, Artificial Sequence
  peptide
SKVLPVPQKAVPYPQ

SEQ ID NO: 137 - protein, 13 aa, Artificial Sequence
  peptide
VLPVPQKAVPYPQ

SEQ ID NO: 138 - protein, 7 aa, Artificial Sequence
  peptide
KAVPYPQ

SEQ ID NO: 139 - protein, 6 aa, Artificial Sequence
  peptide
TDVENL

SEQ ID NO: 140 - protein, 30 aa, Artificial Sequence
  peptide
SVLSLSQSKVLPVPQKAVPYPQRDMPIQAF

SEQ ID NO: 141 - protein, 27 aa, Artificial Sequence
  peptide
SLSQSKVLPVPQKAVPYPQRDMPIQAF

SEQ ID NO: 142 - protein, 25 aa, Artificial Sequence
  peptide
SQSKVLPVPQKAVPYPQRDMPIQAF

SEQ ID NO: 143 - protein, 23 aa, Artificial Sequence
  peptide
SKVLPVPQKAVPYPQRDMPIQAF

SEQ ID NO: 144 - protein, 21 aa, Artificial Sequence
  peptide
VLPVPQKAVPYPQRDMPIQAF

SEQ ID NO: 145 - protein, 15 aa, Artificial Sequence
  peptide
KAVPYPQRDMPIQAF

SEQ ID NO: 146 - protein, 13 aa, Artificial Sequence
  peptide
VPYPQRDMPIQAF

SEQ ID NO: 147 - protein, 16 aa, Artificial Sequence
  peptide
EQQQTEDELQDKIHPF

SEQ ID NO: 148 - protein, 15 aa, Artificial Sequence
  peptide
QQQTEDELQDKIHPF

SEQ ID NO: 149 - protein, 10 aa, Artificial Sequence
  peptide
DELQDKIHPF

SEQ ID NO: 150 - protein, 8 aa, Artificial Sequence
  peptide
LQDKIHPF

SEQ ID NO: 151 - protein, 19 aa, Artificial Sequence
  peptide
SLPQNIPPLTQTPVVVPPF

SEQ ID NO: 152 - protein, 15 aa, Artificial Sequence
  peptide
NIPPLTQTPVVVPPF

SEQ ID NO: 153 - protein, 14 aa, Artificial Sequence
  peptide
IPPLTQTPVVVPPF

SEQ ID NO: 154 - protein, 28 aa, Artificial Sequence
  peptide
VMGVSKVKEAMAPKHKEMPFPKYPVEPF

SEQ ID NO: 155 - protein, 20 aa, Artificial Sequence
  peptide
EAMAPKHKEMPFPKYPVEPF

SEQ ID NO: 156 - protein, 18 aa, Artificial Sequence
  peptide
MAPKHKEMPFPKYPVEPF

SEQ ID NO: 157 - protein, 17 aa, Artificial Sequence
  peptide
APKHKEMPFPKYPVEPF

SEQ ID NO: 158 - protein, 14 aa, Artificial Sequence
  peptide
HKEMPFPKYPVEPF

SEQ ID NO: 159 - protein, 13 aa, Artificial Sequence
  peptide
KEMPFPKYPVEPF

SEQ ID NO: 160 - protein, 12 aa, Artificial Sequence
  peptide
EMPFPKYPVEPF

SEQ ID NO: 161 - protein, 11 aa, Artificial Sequence
  peptide
MPFPKYPVEPF

SEQ ID NO: 162 - protein, 8 aa, Artificial Sequence
  peptide
SVEQKHIQ

SEQ ID NO: 163 - protein, 9 aa, Artificial Sequence
  peptide
SVEQKHIQK

SEQ ID NO: 164 - protein, 9 aa, Artificial Sequence
  peptide
MESTEVFTK

SEQ ID NO: 165 - protein, 10 aa, Artificial Sequence
  peptide
MESTEVFTKK

SEQ ID NO: 166 - protein, 9 aa, Artificial Sequence
  peptide
SAEERLHSM

SEQ ID NO: 167 - protein, 8 aa, Artificial Sequence
  peptide
DIGSESTE

SEQ ID NO: 168 - protein, 11 aa, Artificial Sequence
  peptide
DLISKEQIVIR

SEQ ID NO: 169 - protein, 7 aa, Artificial Sequence
  peptide
EKFQSEE

SEQ ID NO: 170 - protein, 11 aa, Artificial Sequence
  peptide
GKEKVNELSKD

SEQ ID NO: 171 - protein, 9 aa, Artificial Sequence
  peptide
ISKEQIVIR

SEQ ID NO: 172 - protein, 9 aa, Artificial Sequence
  peptide
KDIGSESTE

SEQ ID NO: 173 - protein, 14 aa, Artificial Sequence
  peptide
KDIGSESTEDQAME

SEQ ID NO: 174 - protein, 12 aa, Artificial Sequence
  peptide
KFQSEEQQQTED

SEQ ID NO: 175 - protein, 14 aa, Artificial Sequence
  peptide
KFQSEEQQQTEDEL

SEQ ID NO: 176 - protein, 9 aa, Artificial Sequence
  peptide
KIEKFQSEE

SEQ ID NO: 177 - protein, 9 aa, Artificial Sequence
  peptide
KKIEKFQSE

SEQ ID NO: 178 - protein, 10 aa, Artificial Sequence
  peptide
KKIEKFQSEE

SEQ ID NO: 179 - protein, 17 aa, Artificial Sequence
  peptide
KVPQLEIVPNSAEERLH

SEQ ID NO: 180 - protein, 10 aa, Artificial Sequence
  peptide
LISKEQIVIR

SEQ ID NO: 181 - protein, 9 aa, Artificial Sequence
  peptide
MESTEVFTK

SEQ ID NO: 182 - protein, 10 aa, Artificial Sequence
  peptide
MESTEVFTKK

SEQ ID NO: 183 - protein, 17 aa, Artificial Sequence
  peptide
NREQLSTSEENSKKTVD

SEQ ID NO: 184 - protein, 8 aa, Artificial Sequence
  peptide
SAEERLHS

SEQ ID NO: 185 - protein, 10 aa, Artificial Sequence
  peptide
SAEERLHSMK

SEQ ID NO: 186 - protein, 12 aa, Artificial Sequence
  peptide
SAEERLHSMKEG

SEQ ID NO: 187 - protein, 14 aa, Artificial Sequence
  peptide
SAEERLHSMKEGIH

SEQ ID NO: 188 - protein, 12 aa, Artificial Sequence
  peptide
SEEQQQTEDELQ

SEQ ID NO: 189 - protein, 13 aa, Artificial Sequence
  peptide
SEEQQQTEDELQD

SEQ ID NO: 190 - protein, 10 aa, Artificial Sequence
  peptide
SKDIGSESTE

SEQ ID NO: 191 - protein, 14 aa, Artificial Sequence
  peptide
SKDIGSESTEDQAM

SEQ ID NO: 192 - protein, 15 aa, Artificial Sequence
  peptide
SKDIGSESTEDQAME

SEQ ID NO: 193 - protein, 8 aa, Artificial Sequence
  peptide
SKEQIVIR

SEQ ID NO: 194 - protein, 7 aa, Artificial Sequence
  peptide
STEVFTK

SEQ ID NO: 195 - protein, 12 aa, Artificial Sequence
  peptide
STSEENSKKTVD

SEQ ID NO: 196 - protein, 7 aa, Artificial Sequence
  peptide
SVEQKHI

SEQ ID NO: 197 - protein, 8 aa, Artificial Sequence
  peptide
SVEQKHIQ

SEQ ID NO: 198 - protein, 10 aa, Artificial Sequence
  peptide
SVEQKHIQKE

SEQ ID NO: 199 - protein, 16 aa, Artificial Sequence
  peptide
VPQLEIVPNSAEERLH

SEQ ID NO: 200 - DNA, 23 bp, Artificial sequence
  primer
tgatgcggctcatcagtgccagc

SEQ ID NO: 201 - DNA, 22 bp, Artificial Sequence
  primer
gtagaagtggcctagctcctcg

SEQ ID NO: 202 - DNA, 20 bp, Artificial Sequence
  primer
tgctgactgtggacaactcg

SEQ ID NO: 203 - DNA, 20 bp, Artificial Sequence
  primer
tgcagcgagagcacaaagat

SEQ ID NO: 204 - DNA, 20 bp, Artificial Sequence
  primer
gccacctgctctatgccaag

SEQ ID NO: 205 - DNA, 22 bp, Artificial Sequence
  primer
caggctgtcctagtcaggagat

SEQ ID NO: 206 - DNA, 23 bp, Artificial Sequence
  primer
tgatgcggctcatcagtgccagc

SEQ ID NO: 207 - DNA, 22 bp, Artificial Sequence
  primer
gtagaagtggcctagctcctcg

SEQ ID NO: 208 - DNA, 25 bp, Artificial Sequence
  primer
tggcctctcttggcctccaacttgt

SEQ ID NO: 209 - DNA, 22 bp, Artificial Sequence
  primer
ttgaccaggccttggagagctc

SEQ ID NO: 210 - DNA, 20 bp, Artificial Sequence
  primer
tgctgactgtggacaactcg

SEQ ID NO: 211 - DNA, 20 bp, Artificial Sequence
  primer
tgcagcgagagcacaaagat

(19) United States
(12) Patent Application Publication
(10) Pub. No.: US 2023/0287331 A1
(43) Pub. Date: Sep. 14, 2023

(54) NOVEL LACTIC ACID BACTERIA STRAINS THAT PROMOTE THE ABSORPTION OF CALCIUM - PEPTIDES AND ASSOCIATED PRODUCTS

(71) Applicant: VF BIOSCIENCE, Loos (FR)

(72) Inventors: Benoit CUDENNEC, Villeneuve d'Ascq (FR); François COUTTE, Villeneuve d'Ascq (FR); Pascal DHULSTER, Villeneuve d'Ascq (FR); Djamel DRIDER, Villeneuve d'Ascq (FR); Christophe FLAHAUT, Villeneuve d'Ascq (FR); Marc FREMONT, Loos (FR); Cyril RAVESCHOT, Loos (FR); Maxime VAEREMANS, Loos (FR)

(21) Appl. No.: 17/440,566

(22) PCT Filed: Mar. 9, 2020
(86) PCT No.: PCT/FR2020/050477

§ 371 Date: Sep. 17, 2021

(30) Foreign Application Priority Data
Mar. 20, 2019 (FR) 1902847
Mar. 20, 2019 (FR) 1902851
Mar. 20, 2019 (FR) 1902854

(51) Int. Cl. C12N 1/20; A23L 33/135; A61K 31/59; A61P 3/14

(52) U.S. Cl. CPC C12N 1/205; A23L 33/135; A61K 31/59; A61P 3/14; C12R 2001/225

(57) ABSTRACT

The present invention relates to the Lactobacillus helveticus VF45A strain, mutants and variants thereof capable of reducing the pH of milk to about 3.36 or less after 48 hours of fermentation. The present invention also relates to isolated peptides that may be obtained by fermentation of milk by the aforementioned strain. The present invention also relates to the Lactobacillus helveticus VFH049 strain and mutants and variants thereof capable of producing at least one peptide corresponding to the SEQ ID 44 to SEQ ID NO 86 sequences and/or capable of increasing the intestinal absorption of calcium. The present invention also relates to an isolated peptide and associated products and compositions and the Lactobacillus delbrueckii ssp. bulgaricus VF50b strain and mutants and variants thereof capable of producing at least one peptide corresponding to the SEQ ID 87 to SEQ ID NO 199 sequences and/or capable of increasing the intestinal absorption of calcium.

This application is the U.S. national phase of International Application No. PCT/FR2020/050477 filed Mar. 9, 2020 which designated the U.S. and claims priority to FR Patent Application No. 1902847 filed Mar. 20, 2019, FR Patent Application No. 1902851 filed Mar. 20, 2019 and FR Patent Application No. 1902854 filed Mar. 20, 2019, the entire contents of each of which are hereby incorporated by reference.

### REFERENCE TO SEQUENCE LISTING SUBMITTED ELECTRONICALLY

The content of the electronically submitted sequence listing (Name: 7052_0007_Sequence_Listing.txt; Size: 45.8 kilobytes; and Date of Creation: Mar. 22, 2022) is incorporated herein by reference in its entirety.

The present invention concerns novel lactic acid bacteria strains as well as isolated peptides and mixtures of peptides derived from β-casein and produced by these strains. These peptides are active either as ACE inhibitors or as an agent promoting the calcium absorption.

The present also concerns a food or pharmaceutical composition as well as associated products.

The document WO 2007/096498 A1 describes a novel strain of lactic acid bacterium, more specifically of Lactobacillus helveticus capable of producing, by fermentation of milk, two tripeptides having properties of inhibiting the angiotensin converting enzyme (ACE). These two peptides of IPP and VPP sequences are known to inhibit ACE and therefore allow reducing blood pressure and thus fight against hypertension and the pathologies which result therefrom.

On the other hand, the publication entitled “Bioactive peptide: production and functionality” by H. Korhonen et al and published in International Dairy Journal vol 16, page 945 to 960 in 2006 reviews the peptides obtained by hydrolysis of milk proteins by lactic acid bacteria, and having a biological activity; this publication describes several peptides different from the IPP and VPP peptides, but also known as ACE inhibitors.

Moreover, it is known that in addition to its role in the regulation of blood pressure, ACE is also involved in the mechanisms of bone resorption/formation. Indeed, the article entitled “Inhibition of angiotensin-converting enzyme exerts beneficial effects on trabecular bone in orchidectimozed mice” by X-F Chen, published in the journal Pharmacological reports 70, page 705-711 in 2018, indicates that an ACE inhibitor, namely captopril has a bone preservation activity.

With regard to the calcium absorption, the aforementioned publication by H. Korhonen mentions casein phosphopeptides known to improve the absorption of calcium and zinc. It mentions in particular the Capolac® brand product which contains a casein phosphopeptides (CPP) known to improve the absorption of minerals. With regard to the mechanism of action, it seems that these phosphopeptides are known to bind to calcium, thus making it bioavailable.

The document JP H0641191A describes a Lactobacillus helveticus strain which produces peptides which increase the solubility of calcium. However, this document does not describe any biological activity of the peptides for the absorption of calcium on intestinal cell cultures.

Moreover, the publication “Recent advances in microbial fermentation for dairy and health” by D. Hill et al, published on May 26, 2017 in the journal in F 1000 Research also reports bacteria capable of producing, by proteolysis, bioactive peptides as an ACE inhibitor. It further indicates that a fructo-oligosaccharide can be used as a preventive measure against osteoporosis. The mechanism of action of this fructo-oligosaccharide is as follows: in the belly of the subject, the fructo-oligosaccharide generates by fermentation of the small acid molecules, which lowers the pH in vivo and cause the dissolution of calcium phosphates which would thus be bioavailable.

An insufficient calcium absorption (hypocalcemia) can lead to different symptoms or pathologies such as osteoporosis, arterial hypertension or metabolic syndrome. Indeed, calcium is involved regardless of the age of the patient, in the formation of bones, the solidification of the skeleton, the rigidity of the teeth, muscle contraction and therefore the cardiac contraction, the transmission of the nerve impulses at the synapses, the learning and memorization, the salivary secretion, the cell growth and proliferation. Calcium allows activating many enzymes, releasing several hormones, coagulating the blood and helps balancing the body weight. Hypocalcemia is also linked to various rare pathologies such as autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets and more generally the diseases caused by an abnormality in the regulation of calcemia due to the dysfunction of the parathyroid hormone, of the absorption of vitamin D or the function of the calcium receptor.

To date, osteoporosis is not treated effectively. Thus, bisphosphonates, which are the most widely used treatment, are not very effective in the case of patients who have a low risk of fracture. Prevention through regular practice of a physical activity, a balanced diet and limiting tobacco and alcohol proves be more effective.

The document WO 2007/096498 A1 describes a particular lactobacillus helveticus strain capable of producing ACE inhibitory tripeptides. The publication “characterization of angiotensin-converting enzyme inhibitory activity of fermented milk produced by Lactobacillus helveticus” by Yongfu Chen published in the journal J. Dairy Sci in 2015 describes a Lactobacillus helveticus strain whose fermentate obtained under fermentation conditions other than those used for the strain of the invention inhibits ACE.

The publication entitled “Peptide profiles and angiotensin-I-converting enzyme inhibitory activity of fermented milk products: effect of bacterial strain, fermentation pH and storage time” by Nielsen et al and published in the “International Dairy Journal”, indicates that it is possible to obtain by fermentation of a particular Lactobacillus helveticus strain to obtain a fermentate which inhibits ACE. This document also indicates that the ACE inhibitory activity depends greatly on the pH obtained at the end of the fermentation.

The publication entitled “Technological and probiotic potential of BGRA43 a natural isolate of Lactobacillus helveticus” by Ivana Strahinic published in the journal “frontiers in microbiology” indicates that a Lactobacillus helveticus strain isolated from the human intestine allows fermenting the milk. The fermented milk allows reducing proinflammatory cytokines.

The publication entitled “proteolytic activity of probiotic strain Lactobacillus helveticus M92” by Jasna Beganovic et al, published in the journal “Anaerobe” describes the strain M92 which has a particular proteolytic activity.

The publication entitled “Fermentation characteristics and angiotensin I-converting enzyme-inhibitory activity of Lactobacillus helveticus isolate H9 in cow milk, soy milk and mare milk” by Wang and published in the journal J. Dairy Sci. in 2015 indicates that the H9 strain, which is a Lactobacillus helveticus strain is capable of fermenting different milks and of producing ACE inhibitory tripeptides.

The publication entitled “Production of angiotensin-I-converting enzyme inhibitory peptides in fermented milk fermented by Lactobacillus delbrueckii subsp. bulgaricus SS1 and Lactobacillus lactis subsp. cremoris FTA D1” published in the journal Applied Environmental Microbiology in September 2000 describes a particular strain of Lactobacillus delbrueckii which, by fermentation under particular conditions, produces ACE inhibitory peptides, but no activity on calcium absorption.

The document EP 2 735 616 A mentions a Lactobacillus delbrueckii strain which by fermentation under particular conditions produces peptides which are useful for the diseases caused by reduction in brain activity.

The document WO 2008/002484 describes a Lactobacillus delbrueckii strain capable of producing, by fermentation, peptides useful for the treatment of inflammatory diseases of the colon such as Crohn's disease, ulcerative colitis, irritable bowel syndrome and diarrhea.

An aim of the present invention is to propose a strain of lactic acid bacteria resistant to gastrointestinal digestion and capable of acting in the intestine by acidifying the medium, thereby making the calcium soluble, in order to facilitate its absorption and passage through the intestinal wall.

Another aim of the present invention is to propose a strain of lactic acid bacteria resistant to gastrointestinal digestion and capable of stimulating the calcium absorption by the intestinal cells, by acting directly or indirectly thereon.

Another aim of the present invention is to provide a strain of lactic acid bacteria which has greater growth than certain known lactic acid bacteria and/or which allows a greater acidification of the fermentation medium, in particular of milk.

Another aim of the present invention is to propose a strain of lactic acid bacteria capable of producing new phosphorylated peptides likely to promote the absorption of calcium in the intestine.

Another aim of the present invention is to provide a strain of lactic acid bacteria capable of producing new peptides having ACE inhibitory activity.

Another aim of the present invention is to propose at least one peptide, or even a mixture of peptides which exhibits an ACE inhibitory activity and/or promotes the intestinal absorption of calcium and which preferably, is non-toxic for the intestinal cells.

Another aim of the present invention is to provide at least one peptide, or even a mixture of peptides, which has an ACE inhibitory activity which is greater than the activity of the IPP and VPP peptides.

Another aim of the present invention is to provide a pharmaceutical or food composition capable of treating hypocalcemia and therefore the pathologies which result therefrom.

### First Aspect

According to a first aspect, the invention relates to a Lactobacillus helveticus strain having the prtH2 and prtH3 genes and capable of reducing, by anaerobic fermentation, the pH of milk, in particular cow's milk and in particular skimmed cow's milk to a value substantially equal to or less than 3.36, in particular equal to 3.32 after 48 hours of fermentation at 37° C. By reducing the pH, the strain allows the dissolution of the compounds containing calcium, making the latter absorbable through the intestinal wall. According to this first aspect, the present invention also relates to the Lactobacillus helveticus VF45A strain filed with the CNCM under the order number CNCM-I-5300, the mutants and variants thereof having at least 80% identity, preferably at least 90% identity, preferably at least 95% identity with the genome of said VF45A strain and capable of producing at least one peptide corresponding to the SEQ ID 1 to SEQ ID NO 43 sequences and/or capable of reducing, by anaerobic fermentation, the pH of milk, in particular cow's milk and in particular skimmed cow's milk to a value substantially equal to or less than 3.36, in particular equal to 3.32 after 48 hours of fermentation at 37° C.

According to this first aspect, the invention also relates to the aforementioned strains with reference to the first aspect for their use as a drug and in particular as a drug for the treatment of hypocalcemia or in the treatment of a pathology caused by a deficiency in calcium absorption and in particular selected from arterial hypertension, metabolic syndrome, osteoporosis or in the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor in a subject, in particular a mammal and particularly a human having such a need.

According to this first aspect, the present invention also relates to an isolated peptide which can be obtained by fermentation of milk, in particular cow's milk, by at least one strain according to the first aspect of the invention and selected from the peptides corresponding to the SEQ ID 1 to SEQ ID NO 43 sequences, the peptides having at least 80% identity, in particular at least 90% identity, in particular at least 95% identity with said peptides corresponding to the SEQ ID 1 to SEQ ID NO 19 sequences and capable of inhibiting the ACE and the peptides having at least 80% identity, in particular at least 90% identity, in particular at least 95% identity with said peptides corresponding to the SEQ ID 20 to SEQ ID NO 43 sequences and capable of increasing the intestinal absorption of calcium. The Applicant has indeed demonstrated either the ACE inhibitory activity of the abovementioned peptides—which activity is also linked to good bone health—or the activity of improving the calcium absorption through the intestinal wall due to some of the aforementioned peptides. Thus, the peptides of the invention act on bone health via the calcium absorption through the intestinal wall, and/or via ACE inhibition.

According to this first aspect, the present invention also relates to a mixture of peptides selected from the mixtures comprising or consisting of all peptides of SEQ ID NO 1 to SEQ ID NO 43 sequences, the mixtures comprising or consisting of said peptides of SEQ ID NO 1 to SEQ ID NO 5 sequences, the mixtures comprising or consisting of the 5 peptides of SEQ ID NO 1 to SEQ ID NO 5 sequences and of at least one peptide selected from the peptides corresponding to the SEQ ID 20 to SEQ ID NO 43 sequences, preferably the mixtures containing or consisting of the 5 peptides of SEQ ID NO 1 to SEQ ID NO 5 sequences and the peptides of SEQ ID 20 to SEQ ID NO 43 sequences and the mixtures comprising or consisting of the peptides of SEQ ID 20 to SEQ ID 43 sequences.

These mixtures prove to be active in inhibiting ACE and/or in improving intestinal absorption of calcium.

The present invention also relates to the aforementioned isolated peptides and to the aforementioned mixtures for their use as a drug and in particular as a drug in the treatment of hypocalcemia or in the treatment of a pathology caused by a deficiency in calcium absorption and in particular selected from arterial hypertension, metabolic syndrome, osteoporosis or in the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor, in a subject, in particular a mammal and particularly a human having such a need.

According to this first aspect, the present invention also relates to a food or pharmaceutical composition which contains at least one pharmaceutically acceptable excipient or a medium capable of being ingested and, as active ingredient, at least one peptide selected from the aforementioned isolated peptides with reference to the first aspect and/or a mixture of peptides as mentioned above with reference to the first aspect and/or a strain according to the first aspect of the invention and in particular the VF45A strain.

Advantageously, according to a first embodiment of the first aspect, the food or pharmaceutical composition comprises, at least or only as active peptides, the 5 peptides corresponding to the SEQ ID NO 1 to SEQ ID NO 5 sequences. These peptides are the most active as ACE inhibitors. They are more active than the VPP and IPP tripeptides of the prior art.

According to this first aspect and according to a second embodiment, the food or pharmaceutical composition according to the first aspect of the invention contains as active ingredient or consists of the fermentate obtained by anaerobic fermentation of the optionally skimmed cow's milk by means of a strain according to the first aspect of the invention, said fermentate having optionally undergone an ultrafiltration at a cut-off threshold of 10 kDa or it contains or consists of the fermentate obtained by anaerobic fermentation and optionally after an ultrafiltration at a cut-off threshold of 10 kDa of a protein substrate selected from the protein substrates of plant origin originating from at least one cereal and/or at least one pea and/or at least one fungus and/or at least one nut, the mixtures of at least two protein substrates of plant origin, the milks of animal origin which are optionally thermally sterilized, and/or at least partially skimmed, in particular cow's, goat's or sheep's milk with the exception of yak's milk for the fermentate which is not ultrafiltered at a cut-off threshold of 10 kDa, the mixtures of at least two milks of animal origin and the mixtures of at least one protein substrate of plant origin and at least one milk of animal origin.

The fermentate as mentioned above obtained by anaerobic fermentation and ultrafiltration at a cut-off threshold of 10 kDa is part of the fermentates according to the first aspect of the invention, including the ultrafiltered fermentate obtained from yak's milk.

With regard to the fermentate obtained from cow's milk, it is advantageously obtained according to the temperature and duration conditions indicated in the part relating to the experimental results.

Throughout the present application, the protein substrate of plant origin can be selected from plant milks such as oat milk, almond milk, rice milk, soy milk, spelt milk, hazelnut milk and the mixtures of these milks. Regardless of the milk, it can be thermally sterilized before fermentation by the bacteria according to the invention.

According to a particular embodiment, said fermentate is obtained by fermentation of the aforementioned strain with reference to the first aspect, in milk, in particular cow's milk, which is optionally skimmed and/or thermally sterilized, at a temperature of 40° C. at pH=6 for 72 hours, said fermentate being also optionally subjected to an ultrafiltration at a cut-off threshold of 10 kDa at the end of the fermentation. The ultrafiltered or non-ultrafiltered fermentate proves to be active for ACE inhibition and/or for calcium absorption.

In all aspects of the invention, the food or pharmaceutical composition contains, as active ingredient, the peptide (s) and/or the strain. It can also comprise, also advantageously calcium and/or vitamin D.

In all aspects of the invention, the fact of carrying out an ultrafiltration on the fermentate allows separating the peptides produced by the strain from the proteins. The ultrafiltered fermentate therefore no longer contains proteins. When the ultrafiltered fermentate is subjected to gastrointestinal digestion, there is no generation of other peptides, due to the absence of proteins. The filtrate obtained by ultrafiltration of the fermentate proves to be more active when ingested and digested due to the absence of interference by the peptides from protein digestion.

According to a particular embodiment of the food or pharmaceutical composition according to the first aspect of the invention, it contains a concentration of at least one of said strains and preferably one of said strains, in particular the Lactobacillus helveticus VF45A strain equal to or greater than 10⁷ CFU per gram and/or a concentration of peptides which are identical or different and selected from the peptides of SEQ ID NO 1 to SEQ ID NO 43 sequences, greater than or equal to 18 mg per gram. Preferably, the peptides are the peptides contained in the aforementioned fermentate with reference to the first aspect and in particular the peptides of the SEQ ID NO 1 to SEQ ID No 43 sequences.

According to this first aspect, the present invention also relates to a product selected in particular from the food products, in particular the fermented milks, the optionally fermented fruit and/or vegetable juices, the fermented vegetables and/or fruits, the fermented fungi, cold meats, food preparations based on fish(es) and the dairy products obtained from optionally skimmed milk, in particular from cow, goat, sheep, with the exception of yak's milk, or a mixture of milks. According to the first aspect of the invention, this product contains at least one strain according to the first aspect and/or at least one peptide according to the first aspect and/or at least one mixture of peptides according to the first aspect of the invention and/or a food or pharmaceutical composition according to the first aspect of the invention.

According to this first aspect, the invention also relates to a food supplement adapted for a subject, in particular a human or an animal in particular selected from cats, dogs, poultry, sheep, goats, cattle, reptiles, in particular iguana, which characteristically comprises at least one strain according to claim the first aspect and/or at least one peptide according to the first aspect and/or at least one mixture of peptides according to the first aspect and/or a food or pharmaceutical composition according to the first aspect.

Regardless of the aspect of the invention, the food/pharmaceutical composition or the food supplement can be in the form of a powder, a solution in particular aqueous, a tablet, of a capsule containing the strain in lyophilized form and/or the peptide or the mixture of peptides in the form of powder or solution. The capsule may include an outer shell capable of resisting gastrointestinal digestion so as to deliver its contents into the intestine of the patient. The strain can also be, for example, microencapsulated by a lipid coating and be in the form of an ingestible powder.

In the case of a food supplement according to the first aspect, the tablet, the capsule or any other daily dosage unit form preferably contains at least 0.6 g of active peptides and/or 10⁷ CFU of strain according to the first aspect, optionally as a mixture.

The pharmaceutical composition may preferably be adapted for oral administration.

Throughout the application, the dairy product is not limited according to the invention. It can be a milk drink, a fermented milk, kefir, yogurt, a product based on milk which is coagulated by adding acid or a fresh or leavened cheese, for example.

### Second Aspect

According to a second aspect, the present invention relates to a Lactobacillus helveticus strain having the prtH1 gene and capable of reducing, by anaerobic fermentation, the pH of milk, in particular cow's milk to a value substantially equal to or less than 3.45 and in particular equal to 3.43 after 48 hours at 37° C. and having a parietal hydrophobicity of 40% and resistant to acidity and/or capable of increasing the absorption of calcium by the intestinal cells.

According to this second aspect, the present invention relates to the Lactobacillus helveticus VFH049 strain filed with the CNCM under the order number CNCM-I-5403 and the mutants and variants thereof having at least 80% identity, preferably at least 90% identity, preferably at least 95% identity with the genome of said VFH049 strain and capable of producing at least one peptide corresponding to the SEQ ID 44 to SEQ ID NO 86 and/or capable of reducing, by anaerobic fermentation, the pH of milk, in particular cow's milk to a value substantially equal to or less than 3.45 and in particular equal to 3.43 after 48 hours at 37° C. and/or capable of increasing the absorption of calcium by the intestinal cells.

This strain has proven capable of forming acids which are capable of making calcium soluble in the intestine of a host/patient. It is also capable of producing, by proteolysis of β-casein, active peptides as an ACE inhibitor or allowing a better intestinal absorption of calcium. The strain itself resists digestion and allows, when in contact with intestinal cells, a better absorption of calcium by the latter.

According to this second aspect, the present invention also relates to the strains mentioned with reference to the second aspect and in particular the VFH049 strain or the mutants and variants thereof, for its use as a drug and in particular as a drug in the treatment of hypocalcemia or in the treatment of a pathology caused by a deficiency in calcium absorption and in particular selected from arterial hypertension, metabolic syndrome, osteoporosis or in the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor, in a subject, in particular a mammal and particularly a human having such a need.

According to this second aspect, the present invention also relates to an isolated peptide which can be obtained by fermentation of milk, in particular cow's milk, by at least one strain according to the second aspect of the invention and selected from the peptides corresponding to the SEQ ID 44 to SEQ ID NO 86 sequences, the peptides having at least 80% identity, in particular at least 90% identity, in particular at least 95% identity with said peptides corresponding to the SEQ ID 44 to SEQ ID NO 74 sequences and capable of inhibiting the ACE and the peptides having at least 80% identity, in particular at least 90% identity, in particular at least 95% identity with said peptides corresponding to the SEQ ID 75 to SEQ ID NO 86 sequences and capable of increasing the intestinal absorption of calcium.

According to this second aspect, the invention also relates to a mixture of peptides selected from the mixtures of peptides containing or consisting of all peptides of SEQ ID 44 to SEQ ID 86 sequences, the mixtures comprising or consisting of peptides of SEQ ID NO 44 to SEQ ID NO 58 sequences, the mixtures consisting of peptides of SEQ ID NO 44 to SEQ ID NO 58 sequences and of at least one of the peptides of SEQ ID NO 75 to SEQ ID NO 86 sequences, the mixtures comprising or consisting of the peptides of SEQ ID NO 44 to SEQ ID NO 58 sequences and the peptides of SEQ ID 75 to SEQ ID 86 sequences and the mixtures containing or consisting of the peptides of SEQ ID NO 75 to SEQ ID NO 86 sequences. The peptides of SEQ ID NO 44 to SEQ ID NO 58 sequences are the most active as an ACE inhibitor.

According to this second aspect, the invention also relates to an isolated peptide of the second aspect or the mixture according to the second aspect for its use as a drug and in particular as a drug in the treatment of hypocalcemia or of a pathology caused by a deficiency in calcium absorption and in particular selected from arterial hypertension, metabolic syndrome, osteoporosis or for the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor, in a subject, in particular a mammal and particularly a human having such a need.

According to this second aspect, the present invention also relates to a food or pharmaceutical composition which contains at least one pharmaceutically acceptable excipient or at least one medium capable of being ingested and, as active ingredient, at least one peptide corresponding to the isolated peptides according to the second aspect of the invention, and/or a mixture of peptides according to the second aspect of the invention, and/or a strain according to the second aspect of the invention, in particular the VFH049 strain. Preferably, the food or pharmaceutical composition contains a mixture containing all the peptides of SEQ ID NO 44 to SEQ ID NO 86 sequences or a mixture containing at least or only the peptides corresponding to the SEQ ID NO 44 to SEQ ID NO 58 sequences and optionally the peptides of SEQ ID NO 75 to SEQ ID NO 86 sequences.

According to this second aspect, the food or pharmaceutical composition contains or consists of the fermentate obtained by anaerobic fermentation of the optionally skimmed cow's milk by means of a strain according to the second aspect of the invention, said fermentate having optionally undergone an ultrafiltration at a cut-off threshold of 10 kDa or contains or consists of the fermentate obtained by anaerobic fermentation and optionally after an ultrafiltration at a cut-off threshold of 10 kDa of a protein substrate selected from the protein substrates of plant origin originating from at least one cereal and/or at least one pea and/or at least one fungus and/or at least one nut including almond, the mixtures of at least two protein substrates of plant origin, the milks of animal origin which are optionally thermally sterilized, and/or at least partially skimmed, in particular the cow's, goat's or sheep's milk with the exception of mare's milk for the fermentate which is not ultrafiltered at a cut-off threshold of 10 kDa, the mixtures of at least two milks and the mixtures of at least one substrate of plant origin and at least one milk of animal origin.

According to a particular embodiment of the second aspect of the invention, the food or pharmaceutical composition contains or consists of the fermentate obtained by anaerobic fermentation of the VFH049 strain or of a strain as mentioned above, in milk, in particular cow's milk, optionally skimmed, at a temperature of 40° C. at pH=6 for 72 hours, said fermentate being optionally subjected to an ultrafiltration at a cut-off threshold of kDa. This fermentate contains all peptides of the second aspect of the invention; it is therefore active to promote bone health, both as an ACE inhibitor and as an agent promoting the intestinal absorption of calcium.

Regardless of the embodiment, the fermentate itself, ultrafiltered or not, can be considered as a pharmaceutical or food composition.

According to this second aspect, the aforementioned food or pharmaceutical composition preferably contains a concentration of at least one of said strains and preferably one of said strains, in particular the Lactobacillus helveticus VFH049 strain equal to or greater than 10⁷ CFU per gram and/or a concentration of peptides which are identical or different and selected from the peptides of SEQ ID NO 44 to SEQ ID NO 86 sequences, greater than or equal to 6 mg per gram.

According to this second aspect, the invention also relates to a product selected in particular from the food products, in particular the fermented milks, the optionally fermented fruit and/or vegetable juices, the fermented vegetables and/or fruits, the fermented fungi, cold meats, food preparations based on fish(es) and the dairy products obtained from optionally skimmed milk, with the exception of mare's milk, in particular from cow's, goat's, sheep's milk, or a mixture of milks, which contains at least one strain according to the second aspect of the invention and/or at least one peptide according to the second aspect of the invention and/or at least one mixture of peptides according to the second aspect of the invention and/or a food or pharmaceutical composition according to the second aspect of the invention.

According to this second aspect, the invention also relates to a food supplement adapted for a subject, in particular a human or an animal in particular selected from cats, dogs, poultry, sheep, goats, cattle, reptiles, in particular iguana, which characteristically comprises at least one strain according to the second aspect of the invention and/or at least one peptide according to the second aspect of the invention and/or at least one mixture of peptides according to the second aspect of the invention and/or a food or pharmaceutical composition according to the second aspect of the invention.

By way of example, according to the second aspect of the invention, the pharmaceutical or food composition is in the form of a liquid, in particular of the aforementioned cow's milk fermentate. A volume of 30 ml of this solution containing 0.2 g of the mixture of peptides of the invention is administered daily orally.

### Third Aspect

According to a third aspect, the present invention relates to a Lactobacillus delbrueckii ssp. bulgaricus strain having the prtB gene and capable of reducing, by anaerobic fermentation, the pH of milk, in particular cow's milk, to a value substantially equal to or less than 4.55, and in particular equal to 4.51 after 48 hours at 37° C. and having a parietal hydrophobicity of at least 35% and/or capable of increasing the calcium absorption. Such a strain has proven to be active in the intestine and resistant to gastrointestinal digestion. It is capable of increasing the passage of calcium through the intestinal wall. The acids that it produces also allow solubilizing calcium.

According to this third aspect, the invention relates to the Lactobacillus delbrueckii ssp. bulgaricus VF50b strain filed with the CNCM under the order number CNCM-I-5316 and the mutants and variants thereof having at least 80% identity, preferably at least 90% identity, preferably at least 95% identity with the genome of said VF50b strain and capable of producing at least one peptide corresponding to the SEQ ID 87 to SEQ ID NO 199 sequences and/or capable of reducing, by anaerobic fermentation, the pH of milk, in particular cow's milk to a value substantially equal to or less than 4.55, and in particular equal to 4.51 after 48 hours at 37° C. and/or capable of increasing the intestinal absorption of calcium.

The third aspect also relates to each of the strains as mentioned above for its use as a drug, and in particular as a drug in the treatment of hypocalcemia or in the treatment of a pathology caused by a deficiency in calcium absorption and in particular selected from arterial hypertension, metabolic syndrome, osteoporosis or in the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor, in a subject, in particular a mammal and particularly a human having such a need.

According to this third aspect, the present invention relates to an isolated peptide selected from the peptides corresponding to the SEQ ID 87 to SEQ ID NO 199 sequences, the peptides having at least 80% identity, in particular at least 90% identity, in particular at least 95% identity with said peptides corresponding to the SEQ ID 87 to SEQ ID NO 161 sequences and capable of inhibiting the ACE and the peptides having at least 80% identity, in particular at least 90% identity, in particular at least 95% identity with said peptides corresponding to the SEQ ID 162 to SEQ ID NO 199 sequences and capable of increasing the intestinal absorption of calcium.

According to this third aspect, the invention also relates to a mixture of peptides selected from the mixtures containing or consisting of all peptides of SEQ ID 87 to SEQ ID 199 sequences, the mixtures containing or consisting of the peptides of SEQ ID NO 87 to SEQ ID NO 112 sequences, the mixtures containing or consisting of the peptides of SEQ ID NO 87 to SEQ ID NO 112 sequences and of at least one of the peptides of SEQ ID NO 162 to SEQ ID NO 199 sequences and the mixtures containing or consisting of the peptides of SEQ ID NO 162 to SEQ ID NO 199 sequences.

According to the third aspect, the invention also relates to an isolated peptide mentioned with reference to the third aspect or to a mixture mentioned above with reference to the third aspect for its use as a drug and in particular in the treatment of hypocalcemia or of a pathology caused by a deficiency in calcium absorption and in particular selected from arterial hypertension, metabolic syndrome, osteoporosis or for the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor, in a subject, in particular a mammal, in particular a human having such a need.

According to this third aspect, the invention also relates to a food or pharmaceutical composition which contains at least one pharmaceutically acceptable excipient or at least one medium capable of being ingested and, as active ingredient, at least one peptide according to the third aspect of the invention and/or a mixture of peptides according to the third aspect.

Preferably, the composition contains a mixture containing at least or only as peptides, the peptides corresponding to the SEQ ID NO 87 to SEQ ID NO 161 sequences, or a mixture preferably containing all peptides corresponding to the SEQ ID 87 to SEQ ID NO 199 sequences and/or a strain according to the third aspect.

According to a preferred embodiment of the pharmaceutical or food composition according to the third aspect of the invention, it comprises or consists of the fermentate obtained by anaerobic fermentation of the optionally skimmed cow's milk by means of a strain according to the third aspect of the invention, said fermentate having optionally undergone an ultrafiltration at a cut-off threshold of 10 kDa or it contains or consists of the fermentate obtained by anaerobic fermentation and optionally after an ultrafiltration at a cut-off threshold of 10 kDa of a protein substrate selected from the protein substrates of plant origin originating from at least one cereal and/or at least one pea and/or at least one fungus and/or at least one nut, the mixtures of at least two protein substrates of plant origin, the milks which are optionally thermally sterilized and/or at least partially skimmed, in particular cow's, goat's or sheep's milk, with the exception of yak's milk for said fermentate which is not ultrafiltered at a cut-off threshold of 10 kDa, the mixtures of at least two milks and the mixtures of at least one protein substrate of plant origin and at least one milk of animal origin.

The fermentate obtained by anaerobic fermentation of yak's milk by a strain according to the first or the third aspect of the invention and ultrafiltered at cut-off threshold of 10 kDa respectively is part of the fermentates according to the first and the third aspect of the invention. The fermentate obtained by anaerobic fermentation of mare's milk and ultrafiltered at a cut-off threshold of 10 kDa is part of the fermentates according to the second aspect of the invention.

Advantageously, said food or pharmaceutical composition according to the third aspect of the invention contains a concentration of at least one of said strains and preferably only one of said strains, in particular the Lactobacillus delbrueckii ssp. bulgaricus VF50b strain equal to or greater than 10⁷ CFU per gram and/or a concentration of peptides which are identical or different and selected from the peptides of SEQ ID NO 87 to SEQ ID NO 199 sequences, greater than or equal to 4 mg per gram.

The food or pharmaceutical composition preferably comprises the fermentate obtained by anaerobic fermentation of the strain according to the third aspect of the invention, in milk, in particular cow's milk, which is optionally skimmed, at a temperature of 40° C. at pH=6 for 72 hours, said fermentate being optionally subjected to an ultrafiltration at a cut-off threshold of 10 kDa.

According to this third aspect, the present invention also relates to a product selected in particular from the food products, in particular the fermented milks, the optionally fermented fruit and/or vegetable juices, the fermented vegetables and/or fruits, the fermented fungi, cold meats, food preparations based on fish(es) and the dairy products obtained from optionally skimmed milk, in particular from cow, goat, sheep, with the exception of yak's milk, or a mixture of milks, which characteristically contains at least one strain according to the third aspect of the invention and/or at least one peptide according to the third aspect and/or at least one mixture of peptides according to the third aspect and/or a food or pharmaceutical composition according to the third aspect of the invention.

According to this third aspect, the present invention also relates to a food supplement adapted for a subject, in particular a human or an animal selected in particular from cats, dogs, poultry, sheep, goats, cattle, reptiles, in particular iguana, which characteristically comprises at least one strain according to the third aspect and/or at least one peptide according to the third aspect and/or at least one mixture of peptides according to the third aspect and/or a food or pharmaceutical composition according to the third aspect of the invention.

According to this third aspect, for example, the food or pharmaceutical composition is in the form of a liquid. A volume of 30 ml of this composition containing 0.2 g of peptides are administered daily orally.

### Fourth Aspect

According to a fourth aspect of the invention, the present invention relates to a mixture of at least two strains selected from the Lactobacillus helveticus strains having the prtH2 and prtH3 genes and capable of reducing the pH of the milk by anaerobic fermentation, in particular cow's milk and in particular skimmed cow's milk at a value substantially equal to or less than 3.36, in particular equal to 3.32 after 48 hours of fermentation at 37° C. and in particular the Lactobacillus helveticus VF45A strain filed with the CNCM under the order number CNCM-I-5300, the Lactobacillus helveticus strains having the prtH1 gene, in particular the Lactobacillus helveticus VFH049 strain filed with the CNCM under the order number CNCM-I-5403, capable of reducing the pH of milk by anaerobic fermentation, in particular cow's milk at a value substantially equal to or less than 3.45 and in particular equal to 3.43 after 48 hours at 37° C. and having a parietal hydrophobicity of 40% and resistant to acidity and/or capable of increasing the calcium absorption and the Lactobacillus delbrueckii ssp. bulgaricus strains having the prtB gene, in particular the Lactobacillus delbrueckii ssp. bulgaricus VF50b strain filed with the CNCM under the order number CNCM-I-5316, capable of reducing the pH of milk by anaerobic fermentation, in particular cow's milk to a value substantially equal to or less than 4.55 and in particular equal to 4.51 after 48 hours at 37° C. and having a parietal hydrophobicity of at least 35% and/or capable of increasing the absorption of calcium by the intestinal cells and in particular the mixtures of the three VF45A, VFH049 and VF50b strains.

According to this fourth aspect, the invention relates to a mixture of peptides selected from the mixtures comprising the peptides of SEQ ID NO 1 to SEQ ID NO 199 sequences, the mixtures containing peptides of SEQ ID NO 1 to SEQ ID NO 5 sequences, the peptides of SEQ ID NO 44 to SEQ ID NO 86 sequences and the peptides of SEQ ID NO 87 to SEQ ID NO 112 sequences.

According to this fourth aspect, the invention also relates to an isolated peptide selected from the peptides of SEQ ID NO 1 to SEQ ID NO 199 sequences or a mixture according to the fourth aspect of the invention for its use as a drug and in particular in the treatment of hypocalcemia or in the treatment of a pathology caused by a deficiency in calcium absorption and in particular selected from arterial hypertension, metabolic syndrome, osteoporosis or in the treatment of hypocalcemia induced by a pathology selected from autoimmune hypoparathyroidism, vitamin-D dependent hypocalcemic rickets, progressive bone heteroplasia, isolated hypoparathyroidism, syndromic hypoparathyroidism, pseudo-hypoparathyroidism and hypo-deficiency rickets, the diseases caused by an abnormality in the regulation of calcemia due to the dysfunction of the parathyroid hormone, of the absorption of vitamin D or the function of the calcium receptor.

According to this fourth aspect, the invention relates to a food or pharmaceutical composition which comprises at least one pharmaceutically acceptable excipient or at least one medium capable of being ingested and at least one mixture of strains according to the fourth aspect of the invention and/or at least one mixture of peptides according to the fourth aspect of the invention and/or at least the fermentate obtained by fermentation of the mixture of strains according to the fourth aspect of the invention, in milk, in particular cow's milk, which is optionally skimmed, at a temperature of 40° C. at pH=6 for 72 h, said fermentate being optionally subjected to an ultrafiltration at a cut-off threshold of 10 kDa.

According to this fourth aspect, the invention relates to a food or pharmaceutical composition which contains a pharmaceutically acceptable excipient or a medium capable of being ingested and at least one peptide as mentioned above or a mixture of peptides according to the fourth aspect of the invention and/or a mixture of strains according to the fourth aspect of the invention.

According to this fourth aspect, the invention also relates to a product selected from food products, in particular the fermented milks, the optionally fermented fruit and/or vegetable juices, the fermented vegetables and/or fruits, the fermented fungi, cold meats, food preparations based on fish(es) and the dairy products obtained from optionally skimmed milk, from cow, goat, sheep or a mixture of milks which contains a mixture of strains according to the fourth aspect of the invention and/or at least one mixture of peptides according to the fourth aspect of the invention and/or a food or pharmaceutical composition according to the fourth aspect of the invention.

Advantageously, the food or pharmaceutical composition contains a concentration of said strains equal to or greater than 10⁷ CFU per gram of product and/or a concentration of peptides which are identical or different greater than or equal to 4 mg per gram.

### Definitions

The term “milk”, in the absence of precision, refers to cow's milk.

The term “yogurt” refers to a dairy product produced by fermentation by adding Streptococcus thermophilus and Lactobacillus delbrueckii subsp. bulgaricus lactic acid bacteria.

The term “fermented milk” refers to a dairy product obtained by coagulation of caseins following the addition of a lactic ferment in milk.

The term “fermentate” designates the supernatant obtained at the end of the fermentation.

The terms “increase calcium absorption” and other related terms refer to the ability of the strain or peptides to increase the overall passage of calcium through the intestinal wall of a mammal (between the cells of the latter due to the increase in the expression of the cId-2 gene, for example), or to increase the fluorescence in Caco-2 cells as indicated in the experimental part (greater passage of calcium through the cells of the intestinal wall) or to increase the expression of at least one gene selected from trpv6, and vdr in intestinal cells as indicated in the experimental part of the present application (better absorption by the cells themselves).

The terms “resistant to acidity” refer to class 3 indicated in the experimental part of the present application with reference to the resistance to acidity.

The term “probiotic” refers to a microorganism capable of surviving in the intestine of a host, in particular a human, and of interacting with the cells of the intestinal wall and/or of interacting with the other microorganisms living in the intestine of the host and of exerting a beneficial effect on the health of the host.

The term “hypocalcemia” designates a calcium concentration in the blood, corrected with the albumin level which is less than 2.20 mmol/L.

The term “active ingredient” means that the considered ingredient (peptide, mixture of peptides and/or strain) is contained in an amount/concentration allowing obtaining a technical effect at least in vitro on the inhibition of ACE and/or on the calcium absorption.

The term “subject” refers to a human or an animal, in particular a mammal (in particular, dog, cat, poultry, sheep, cattle, goat or reptile).

The term “treatment” encompasses both the preventive (prophylactic) treatment and the treatment allowing obtaining an improvement of at least one symptom of the considered pathology.

The term “percentage identity” refers to the percentage of identical amino acid residues between the sequence to be compared and the reference sequence, by aligning the entire sequence compared to the reference sequence. The protein sequence which presents a % identity with the reference sequence can thus comprise insertions or deletions. The percentage identity is calculated by determining the number of positions at which an identical amino acid residue is observed for the two compared sequences, then by dividing the number of positions for which there is identity between the two nucleobases, or between the two amino acid residues, by the total number of positions in the comparison window, then by multiplying the result per hundred in order to obtain the percentage identity in nucleotides or in amino acids of the two sequences therebetween.

The peptides defined by their % identity can thus comprise one or more deletion(s) and/or one or more substitution(s), the insertion or the substitution by a dextrorotatory amino acid, the replacement of the terminal carboxylic acid group and/or of the terminal amine group by a protective group, for example, the introduction of a retro type bond (that is to say a peptide bond —NH—CO— resulting from the reaction between the amine function of the first amino acid in the sequence with the carboxylic acid function of the following amino acid in the same sequence) or the introduction of a retro-inverso type bond (that is to say the introduction of a peptide bond of the aforementioned retro type with an amino acid in the reverse configuration than that of the amino acid of the reference sequence) or comprise a longer amino acid chain.

Regardless of the aspect of the invention, the peptides may or may not be phosphorylated.

A pharmaceutical composition is defined as being a composition containing an active ingredient and a pharmaceutically acceptable excipient and in particular selected from water, oils, alcohols, in particular ethyl alcohol, vegetable oils, cyclodextrins, starch, maltodextrin, lactose, sucrose, propylene glycol and physiological saline.

The term “consisting” when it is used with reference to one or more peptides indicates that the mixture or the composition contains as peptide(s) only the peptides mentioned with the exception of other peptides, but other compounds or constituents such as solvents, vitamins, proteins or others may be present.

A “medium capable of being ingested” means any mixture or any substance regardless of its physical phase which can be ingested and digested without causing any problem to the digestive tract. The pharmaceutically acceptable excipients and in particular the examples mentioned in the present application are media capable of being ingested.

Throughout the present application, the sequences are conventionally indicated in the direction of their N-terminal end to their C-terminal end.

Throughout the present application, the VFH049 strain can also be referred to as VF49d.

Throughout the invention, the term “strain” refers without distinction to the living strain, that is to say capable of being developed in a suitable nutrient medium and to the inactivated strain, that is to say dead and therefore unable to be developed in an environment suitable for its development.

### FIGURES

FIG. 1 represents electron microscope photographs of each of the three strains of the invention; the leftmost photograph represents the VF45A strain, the middle one the VFH049 strain and the right one the VF50b strain.

FIG. 2 represents the map obtained by PCA which assesses the correlations between the proteolytic activity on agar-milk, the growth during a fermentation in liquid medium (milk), the pH at the end of the fermentation, the amount of produced peptides and 5 other criteria related to the analysis of the chromatographic profiles of the peptides produced by the strains (apparent molecular weight distribution);

FIG. 3 represents the IC₅₀ in mg/mL for the ACE inhibitory activity of the control and the crude fermentates obtained by fermentations of the VF45A and VFH049 strains;

FIG. 4 represents the IC₅₀ in mg/mL for the ACE inhibitory activity of the control and the fermentates obtained by fermentations of the VF45A and VFH049 strains and having undergone an ultrafiltration;

FIG. 5 represents the calcium absorption rate (fluorescence emission/basal emission ratio) of the control and the crude fermentates obtained by fermentations of the VF45A and VFH049 strains;

FIG. 6 represents the calcium absorption rate (fluorescence emission/basal emission ratio) of the control and the fermentates obtained by fermentations of the VF45A and VFH049 strains and having undergone an ultrafiltration;

FIG. 7 represents the mRNA level of the trpv6 gene relative to that of the PBS condition of the crude fermentates obtained by fermentations of the VF45A and VFH049 strains;

FIG. 8 represents the mRNA level of the trpv6 gene relative to that of the PBS condition of the fermentates obtained by fermentations of the VF45A and VFH049 strains and having undergone an ultrafiltration;

FIG. 9 represents the percentages of parietal hydrophobicity obtained for the strains tested depending on their acidity tolerance class.

FIG. 10 represents the mRNA level relative to the control of the vdr, trpv6 and cId-2 genes obtained after 24 hours of contact with the VFH049 strain and the VF50b strain with HT-29 MTX cells.

### EXPERIMENTAL PART

### Isolation and Characterization of the Strains

The three strains of the invention have been extracted from different dairy products.

The samples of each dairy product are collected in sterile tubes, kept at 4° C. for a maximum of two days before analysis. The samples are subjected to a series of dilutions in a saline buffer (0.85% NaCl solution), then spread on MRS-agar plates (De Man, Rogosa and Sharpe). The plates are incubated for 48 hours at 37° C. in anaerobic medium. The morphologically distinct colonies are separated, subcultured on plates of the same medium for purification. In order to ensure the purity of the isolates, the re-spreading on plate is repeated at least three times.

The Table 1 below indicates the dairy products from where the strains were extracted. All these dairy products originate from Mongolia and were prepared in the fall.

Optical microscopic observations are performed to determine the shape of the bacteria. These observations are visible in FIG. 1. The Gram of each strain is conventionally determined by the Gram staining technique and microscope observation. The search for catalase is performed for each strain in a conventional manner by sampling each strain on an agar medium and contacting with hydrogen peroxide.

The results are collected in Table 1 below.

TABLE 1

| Source | | | | | |
|---|---|---|---|---|---|
| dairy | | | | | |
| product | strain | catalase | Gram | form | Species |
| Yogurt | VF45A | negative | + | bacillus | L. |
| (yak's | | | | | helveticus |
| milk) | | | | | |
| Fermented | VF49d | negative | + | bacillus | L. |
| mare's | | | | | helveticus |
| milk | | | | | |
| Yogurt | VF50b | negative | + | Curved | L. |
| (yak's | | | | bacillus | delbrueckii |
| milk) | | | | | ss |
| | | | | | bulgaricus |

### Deposit of the Strains

The Lactobacillus helveticus VF45A strain was filed by the Applicant on Mar. 29, 2018 with the CNCM (National Collection of Cultures of Microorganisms), cis at 25 Docteur ROUX street 75724 Paris Cedex 15 under the order number CNCM-I-5300 (filing according to the Budapest Treaty).

The Lactobacillus helveticus VFH049 strain was filed by the Applicant, on Feb. 19, 2019 with the CNCM (National Collection of Cultures of Microorganisms), cis at 25 Docteur ROUX street 75724 Paris Cedex 15 under the order number CNCM-I-5403 (filing according to the Budapest Treaty). In the remainder of the application, this strain is also called VF49d.

The Lactobacillus delbrueckii ssp. bulgaricus VF50b strain was filed by the Applicant, on Apr. 20, 2018 with the CNCM (National Collection of Cultures of Microorganisms), cis at 25 Docteur ROUX street 75724 Paris Cedex 15 under the order number CNCM-I-5316 (filing according to the Budapest Treaty).

### Sequence of the Genome Encoding the 16S Ribosomal RNA

The determination of the sequence encoding the 16S ribosomal RNA of the strains confirmed that the VF45A and VFH049 strains are indeed strains of the Lactobacillus helveticus species.

Likewise, the analysis of the genome to confirm that the VF50b strain is indeed a strain of the Lactobacillus. delbrueckii ssp. Bulgaricus species.

### PCR Detection of Genes Encoding Parietal Proteases

The identification of parietal proteases present in the selected strains is carried out by PCR detection of the genes encoding these enzymes. The bacterial cells are recovered from the skimmed milk cultures, the bacterial DNA is then isolated using a wizard genomic DNA purification kit (Promega, Madison, United States). The CNRZ32 CIRM-BIA 103 strain of Lactobacillus helveticus (supplied by the International Center for Microbial Resources—Food Related Bacteria, CIRM-BIA, INRA, Rennes, France) was used as a control for the Lactobacillus helveticus genes. This (CNRZ32) strain, is known to have the 4 prtH genes (prtH1 to prtH4).

Each strain is tested for 5 pairs of primers corresponding to 5 different genes, namely, prtB, prtH1, prtH2, prtH3 and prtH4. The primers described by Hou et al., 2015 are used for the detection of prtB in Lactobacillus delbrueckii. Concerning the Lactobacillus helveticus strains, the primers described by Genay et al., 2009 are used for the detection of the prtH1 and prtH2 genes and finally those described by Broadbent el al., 2011 for the detection of prtH3 and prtH4.

The PCR reactions are carried out in a final volume of 25 μL comprising 12.5 μL of PCR Master Mix (2) (ThermoFisher Scientific, Waltham, United States), 2 μL of each primer (12.5 μM), 2 μL of extracted bacterial DNA (about 200 ng. μL⁻¹) and 6.5 μL of H₂O. The PCR cycles are performed in a thermocycler labcycler (SensoQuest, Göttingen, Germany). After a first denaturation at 95° C. for 5 min, 30 cycles of PCR are repeated successively, one cycle consists of a denaturation at 95° C. for 30 sec, hybridization for 1 min at the required hybridization temperature for each primer and an elongation at 72° C. for 30 sec (4 min for the prtH4 gene). A last elongation at 72° C. for 10 min takes place after the 30 PCR cycles. The PCR products are analyzed by electrophoresis on agarose gel at 1% (m/v) prepared in a TBE buffer (Tris, Borate, EDTA). The revelation of the DNA fragments is made by adding, in the gel, 0.008% (v/v) of GelRed® (10,000×) (Biotium, Fremont, United States). Ten μL of PCR products are mixed with 4 μL of loading buffer (6ₓ) (ThermoFisher Scientific, Waltham, United States) before being deposited on the agarose gel. The O'GeneRuler DNA Ladder Mix (ThermoFisher Scientific, Waltham, United States) mixture is used as a size marker. The migration takes place for 45 min at a constant voltage of 100 V. The amplification products are revealed with Gel Doc™ (Bio-Rad, Hercules, United States).

The results are shown in Table 2 below. The prtB gene is detected for the VF50b strain of Lactobacillus delbrueckii, but not in the VF45A and VFH049 strains of Lactobacillus helveticus. The prtH1 gene is detected for the VFH049 strain of Lactobacillus helveticus and in the control. Interestingly, other strains (not represented) were tested and it proves that all Lactobacillus helveticus strains having the prtH1 gene were isolated from fermented mare's milk. The prtH2 and prtH3 genes are detected in the VF45A strain of Lactobacillus helveticus and in the control. The prtH4 gene is detected in the control, but not in the tested strains.

TABLE 2

| [symbol] | [symbol] | prtH1 [symbol] | prtH2 [symbol] | prtH3 [symbol] | [symbol] | [symbol] | [symbol] |
|---|---|---|---|---|---|---|---|
| VF50b [symbol] | L. | [symbol] | [symbol] | [symbol] | [symbol] | + [symbol] | Yak’s [symbol] |
| | [symbol] | | | | | | |
| CNRZ32 [symbol] | L. | + [symbol] | + [symbol] | + [symbol] | + [symbol] | [symbol] | CIRM·(INRA) [symbol] |
| | [symbol] | | | | | | |
| VF49d [symbol] | L. | + [symbol] | [symbol] | [symbol] | [symbol] | [symbol] | Mare·s milk [symbol] |
| | [symbol] | | | | | | |
| VF45A [symbol] | L. | [symbol] | + [symbol] | + [symbol] | [symbol] | [symbol] | Yak’s milk [symbol] |
| | [symbol] | | | | | | |

[symbol]

indicates data missing or illegible when filed

### Determination of the Proteolytic and Fermentation Activity of the Strains

The used tests cover both fermentation capacity (speed of growth, acidification) and proteolytic activity (ability to hydrolyze proteins). Indeed, the two aspects are largely linked, the ability of bacteria to grow indeed depends on their ability to hydrolyze proteins (since they need the amino acids released during hydrolysis to ensure their growth). It is also already known that acidification of the medium and hydrolysis of proteins are correlated.

1) Laboratory Scale—Fermentation without pH Control

### Determination of the Proteolytic Activity According to the Milk Agar Method

Each strain is cultured in agar-milk wells; the appearance of a halo around the well reflects a proteolysis. The measurement of the diameter of the halo allows assessing the proteolytic capacities of the strain. Agar-milk is prepared by mixing, in water, a powder of skimmed milk (Sigma-Aldrich, St. Louis, United States) at a concentration of 5% (m/v) supplemented with 1.5% (m/v) agar. After sterilization at 110° C. for 10 min, the milk is poured into Petri dishes. 4-mm diameter wells are then formed in the agar. The strain is cultivated in liquid MRS medium (Agar of Man, Rogosa, Sharpe) for 48 h at 37° C. under anaerobic condition. The bacterial cells are then washed twice in MRS medium then finally re-suspended in a volume of MRS medium allowing obtaining an OD₆₀₀ equal to 1 (Prim spectrophotometer, Secomam, Aqualabo Group, Champigny, France). A volume of 20 μL of the bacterial suspension is deposited in the agar-milk wells. The inoculated plates are then incubated for 72 hours at 37° C. under anaerobic conditions. The proteolytic activity of the strains is quantified by measuring the diameter (expressed in mm) of the halos that appear around the wells after incubation.

### Skimmed Milk Cultures: Growth, pH

From a bacterial culture in liquid MRS medium, a pre-culture of 5 mL is carried out in a medium composed of 100% UHT skimmed milk (Cora, Paris, France), for 72 hours at 37° C., in anaerobic condition. The strain is then inoculated into a final volume of 30 mL of the same medium, with an initial OD₆₀₀ of 0.3. The cultures take place for 48 hours at 37° C. in anaerobic condition. As a control, the same non-inoculated milk medium (NI Milk) is incubated under the same conditions. At the end of the fermentation, the acidity of the medium is assessed by measuring the pH by a pH meter (Mettler Toledo, Greifensee, Switzerland). For assessing the bacterial growth, a direct measurement of the OD cannot be carried out due to the opacity of the medium. The phenomenon of casein coagulation after fermentation is also a problem, caseins precipitating as soon as the pH is below 4.6. A prior protocol for recovering bacterial cells from the milk culture is therefore carried out. A volume of 1 mL of culture is diluted (1/10) in a 2% (m/v) EDTA solution at pH 12, this is in order to completely re-suspend the precipitated caseins. The cells are then recovered by centrifugation at 13,400 rpm for 10 min. This protocol is repeated a second time and the obtained bacterial pellet is resuspended in 1 ml of PBS buffer. The measurement of OD₆₀₀ is carried out from this suspension, the blank is obtained by carrying out the same protocol with non-inoculated milk.

The cell growth and the pH were measured after 48 hours of incubation in skimmed milk. The Lactobacillus helveticus strains show a very rapid growth, reaching an average OD₆₀₀ of 2.7 while the Lactobacillus delbrueckii strains reach only 0.85 on average.

The acidification of the inoculated milk was compared to that of the control (NI Milk). The initial pH of the non-inoculated milk was 6.5, it reached 6.45 after 48 hours of incubation. In almost all cases the of Lactobacillus helveticus strains led to a decrease in pH of 3 units, reaching an average of 3.68, while the pH of the samples inoculated with the Lactobacillus delbrueckii strain decreased by 2 pH units at most, reaching an average of 4.57. In all cases, the fermentation resulted in the coagulation of the milk because the caseins precipitate when the pH drops below 4.6.

Measurement of the Amounts of Produced Peptides and their Molecular Weight Distribution

The peptides produced during the fermentation of the strains are analyzed by two methods. The peptide concentrations are assessed first by assaying with the Folin-Ciocalteu (FC) reagent; the peptides are also analyzed depending on their apparent molecular weight distribution by size exclusion chromatography (SEC).

Extraction and Purification of the Produced Peptides

From the skimmed milk cultures, a 4 mL sampling is carried out then mixed with a volume of 400 μL of a solution of trichloroacetic acid (TCA) at a concentration of 10% (m/v) to reach a final TCA concentration of 1%.

The addition of such a TCA concentration allows the precipitation of high molecular weight proteins. After a centrifugation at 10,000 rpm for 10 min, the supernatant containing the peptides is recovered.

The peptides are then purified from the supernatant in order to remove the TCA, the sugars, and the salts. The purification is carried out by solid phase extraction (SPE), the principle of this method is a separation of the compounds of a mixture by selective adsorption on a solid phase. Bond Elut C₁₈ (1000 mg) micro-columns (Agilent Technologies, Santa Clara, United States) are used; these are columns composed of a solid phase of silica grafted with C₁₈ groups allowing the retention of hydrophobic compounds. The columns are balanced by passing a minimum volume of 10 mL of a 100% acetonitrile (ACN) solution supplemented with 0.1% (v/v) trifluoroacetic acid (TFA). After rinsing the column with a H₂O solution containing 0.1% (v/v) TFA, a volume of 4 mL of supernatant containing the extracted peptides is then loaded onto the column. A new rinse with H₂O supplemented with 0.1% TFA allows eluting all compounds which are not retained by the column. The retained peptides are finally eluted in a volume of 2 mL of an ACN solution at 80% (v/v), 20% (v/v) H₂O and containing 0.1% TFA. The samples are then stored at −20° C. until analysis.

Folin-Ciocalteu Reagent Assay

The extracted peptides are quantified by an assay with the Folin-Ciocalteu (FC) reagent. It is a sodium tungstate and molybdate solution prepared in phosphoric and hydrochloric acids. The complex of phosphotungstic acid and phosphomolybdic acid of yellow color is reduced by tyrosine residues, tryptophan, cysteine or else by peptide bonds in an alkaline medium to give a blue color. The appearance of the blue color, proportional to the peptide concentration, is followed by spectrophotometry. For the assay, the reaction is carried out in a final volume of 800 μL comprising 200 μL of extracted peptides, 500 μL of a solution of sodium carbonate (NaHCO₃) at 500 mM and 100 μL of Folin-Ciocalteu reagent (Sigma-Aldrich, St Louis, United States). The reaction is incubated at 37° C. in the dark for 30 min, then the OD at 750 nm of the mixture is measured by a Prim spectrophotometer (Secomam, Aqualabo Group, Champigny, France). The blank is produced from the solution used to elute the peptides from the SPE columns (80% ACN, 20% H₂O, 0.1% TFA). The concentration in the samples is determined by a range of standards of a commercial peptide solution (peptide digest assay standard, ThermoFisher Scientific, Waltham, United States).

Size Exclusion Chromatography Analysis

The purpose of size exclusion chromatography (SEC) is the separation of the compounds of a mixture as a function of their hydrodynamic sizes or volumes. In this study, the SEC allows analyzing the apparent molecular weight distribution of the peptides extracted after fermentation. The column used for the separation of the peptides is a Superdex Peptide 10/300 GL (10×300-310 mm, 13 μm, GE Healthcare, Little Chalfont, United Kingdom) connected to a system of AKTA Protein purification (GE Healthcare, Little Chalfont, United Kingdom). A volume of 25 μL of sample is eluted under isocratic condition in a solvent composed of 30% (v/v) CAN, 70% (v/v) H₂O and 0.1% (v/v) TFA, at a flow rate of 0.5 mL/min for 60 min. A UV detector at 214 nm allows the detection of the peptide bonds. The amount of peptides in the samples is analyzed by integration of the profiles obtained by SEC. Two ranges of molecular weights were selected for the integrations, the first relates to the peptides of apparent molecular weight greater than 1,700 Da (called HMWP, high molecular weight peptides) and the second relates to the peptides of apparent molecular weight less than 1,700 Da (called LMWP, low molecular weight peptides). The amount of peptides belonging to each size class is expressed either as a percentage of the total area of the chromatogram (% HMWP and % LMWP), or as a percentage of the area of the chromatogram obtained for the non-inoculated milk control condition (% HMWP/NI Milk and % LMWP/NI Milk).

The concentration measurements using the FC reagent showed an increase in the amount of peptides after 48 h, with concentrations greater than 4 g/L in all cases, compared to 2/06 g/L in the non-inoculated sample.

The Lactobacillus helveticus VF45A strain is one of the most effective strains with a final peptide concentration greater than 6 g/L (see Table 3 below).

The molecular weight of the generated peptides was analyzed by SEC as mentioned above. The amount of peptides belonging to each size class (less than or greater than

TABLE 3

| | ø | | | FC | | | | | |
|---|---|---|---|---|---|---|---|---|---|
| | agar | Acid- | | reagent | Total | | | | |
| | milk | ification · | Growth | assay | area | | | % HMWP/ | % LMWP/ |
| Strain | (mm) [symbol] | (pH) [symbol] | (OD) [symbol] | (g/L) | (mAU) [symbol] | % HMWP | % LMWP | CTLF [symbol] | CTLF [symbol] |
| NISM [symbol] | x [symbol] | 6.45 ± | x [symbol] | 2.06 ± | 347.51 ± | 86.62 ± | 14.39 ± | 100 [symbol] | 100 [symbol] |
| | | 0.05 [symbol] | | 0.62 [symbol] | 42.8 [symbol] | 2.72 [symbol] | 2.72 [symbol] | | |
| VF50b [symbol] | 30 ± 1.5 | 4.51 ± | 0.973 ± | 4.89 ± | 114.70 ± | 58.12 ± | 40.90 ± | 21.12 ± | 140.37 ± |
| | | 0 | 0.199 [symbol] | 0.3 [symbol] | 12.5 [symbol] | 6.22 [symbol] | 4.26 [symbol] | 1.81 [symbol] | 28.22 [symbol] |
| VF4SA [symbol] | 32 ± 4 [symbol] | 3.32 ± | 3.57 ± | 6.16 ± | 289.95 ± | 40 ± | 60.1 ± | 37.35 ± | 514.7 ± |
| | | 0.04 [symbol] | 0.12 [symbol] | 1.32 [symbol] | 52 [symbol] | 3.74 [symbol] | 3.77 [symbol] | 11.95 [symbol] | 86.67 [symbol] |
| VF49d [symbol] | 35 ± 4.3 [symbol] | 3.43 ± | 2.72 ± | 4.46 ± | 225.08 ± | 58.56 ± | 42.19 ± | 43.87 ± | 263.68 ± |
| | | 0.01 [symbol] | 0.05 [symbol] | 0.46 [symbol] | 118.3 [symbol] | 14.51 [symbol] | 14.63 [symbol] | 29.24 [symbol] | 77.95 [symbol] |

[symbol]

indicates data missing or illegible when filed

1,700 Da) is expressed either as a percentage of the total area of the chromatogram (% HMWP and % LMWP), or as a percentage of the area of the chromatogram obtained for the non-inoculated milk control condition (% HMWP/NI Milk and OR LMWP/NI Milk). The inoculation and the fermentation lead to an increase in low molecular weight peptides relative to the control (1.5 times on average for the Lactobacillus delbrueckii strains, 4.2 times on average for the Lactobacillus helveticus).

The results obtained for each strain are collected in Table 3 below.

The main component analyzes (PCA) are carried out with the R software (R core team, 2016, Vienna, Austria), on the R Commander package and its FactorMineR plug-in.

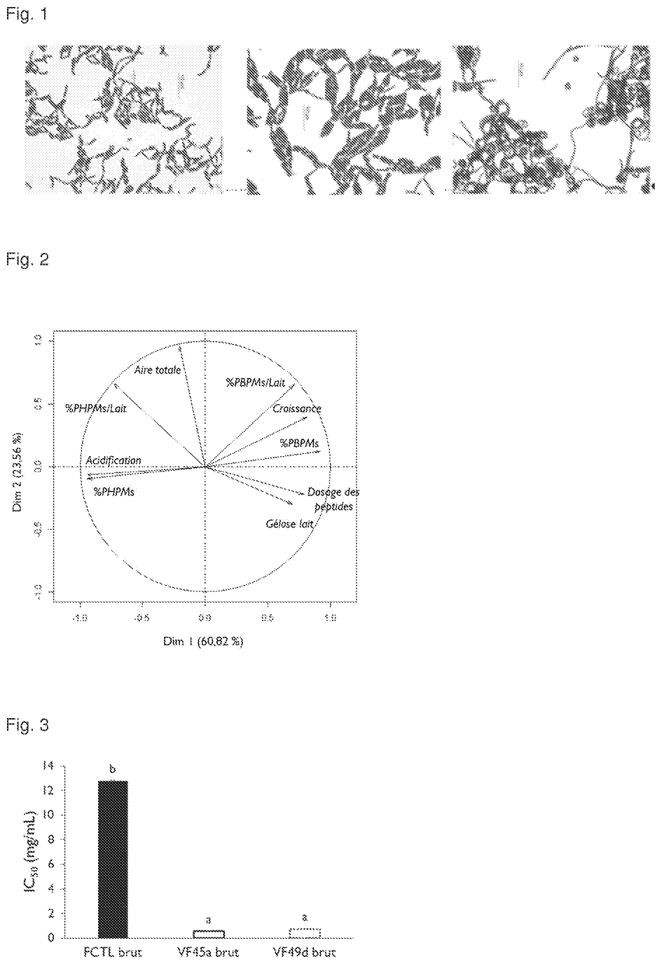

A PCR analysis took into account 9 quantitative parameters: the proteolytic activity on agar-milk, the growth during a fermentation in liquid medium (milk), the pH at the end of the fermentation, the amount of produced peptides, and 5 other criteria related to the analysis of the chromatographic profiles of the peptides (apparent molecular weight distribution). FIG. 2 shows the obtained map. The main components Dim1 and Dim2 represent 60.82% and 23.56% of the total variance, respectively. A positive correlation is observed between the pH value and the high molecular weight peptides. In addition, growth is correlated with a series of variables linked to the production of peptides (quantification by FC reagent, % LMWP, % LMWP/NI Milk) and the diameter of the proteolysis halo. The pH value and the high molecular weight peptides are negatively correlated with variables such as growth, proportion of LMWP and halo of proteolysis.

### 2) Production of the Peptides at Controlled pH, Ultrafiltration, Identification of the Peptides

### Fermentation in a Bioreactor

Each strain is cultured in a 500 mL bioreactor (MiniBio 500, my-Control, Applikon Biotechnology, Delft, the Netherlands). The medium consists of skimmed milk (10 g/L (see previous paragraph), autoclaved for 30 min at 110° C. The strain is inoculated at an OD₆₀₀ equal to 0.3. The fermentation is carried out for 72 hours at a constant temperature of 40° C. with stirring at 300 rpm in a final volume of 330 mL. The anaerobic condition is obtained by injecting N₂ into the bioreactor at a flow rate of 20 mL/min. Throughout the experiment, the pH is maintained at 6 thanks to solutions of HCl (1 M) and NaOH (3 M), the supply is carried out by means of two peristaltic pumps located on the control unit of the bioreactor. Samples of approximately 5 mL are taken under aseptic conditions at 0, 2, 17, 24, 41, 48, 65 and 72 hours of fermentation for analyzing the growth of the bacterial strains as well as the evolution of the concentration of peptides. The latter is assessed by an FC reagent assay after a TCA precipitation. A volume of 1 mL of fermentate is diluted (1/10) in a solution of 2% (m/v) EDTA at pH 12 then centrifuged at 13,400 rpm for 10 min, and 25 μL of supernatant are analyzed by SEC. The bacterial pellet is resuspended in 1 mL of PBS buffer for measuring the OD₆₀₀ in order to assess the bacterial growth. After 72 hours of fermentation, the entire fermentate is recovered then centrifuged at 10,000 rpm for 10 min, the supernatant is then stored at −20° C. until analysis. The concentration of dry matter in the fermentates is measured in a desiccator (XM60, Precisa, Poissy, France) and is always equal to 100 g/L. In order to be able to confirm the impact of the bacterial fermentation on the properties of the final product, a control is carried out (CTLF). This is the fermentation medium incubated under the same conditions but not inoculated. After 72 hours of incubation, this medium is centrifuged and stored like the other fermentations.

### Identification of the Peptides Produced During the Fermentation at Controlled pH

In order to remove unhydrolyzed proteins, a portion of the crude fermentate is fractionated by means of an ultrafiltration membrane. The membrane is a Hydrosart cassette (Sartocon® Slice Hydrosart® Cassette, Sartorius, Göttingen, Germany) of 0.1 m² with a cut-off threshold of 10 kDa connected to a Sartocon® Slice 200 Holder system (Sartorius, Göttingen, Germany). The supply of the fermentate is carried out by a peristaltic pump and the retentate pressure is maintained at about 1 bar during filtration. Thus, the ultrafiltration permeate (UFP) containing the molecules less than 10 kDa is separated from the retentate (UFR). The two sub-fractions are then stored at −20° C. until analysis. The ultrafiltration permeates are dried to 10% of the initial volume by centrifugal evaporation (miVac, Gene Vac, Ipswich, United Kingdom) for 15 h at 40° C. The dry matter concentration in the products (fermentate and ultrafilter fermentate) is then measured in a desiccator (XM60, Precisa, Poissy, France).

The peptides purified by SPE from the culture supernatants (not ultrafiltered) are dried by centrifugal evaporation (miVac, Gene Vac, Ipswich, United Kingdom) for 2 h at 40° C. then re-suspended in 100 μL of H₂O containing 0.1% (v/v) TFA and centrifuged for 10 min at 8,000 rpm. Ten microliters of sample are injected onto a C₁₈-Kinetex column (150×4.6 mm, 2.6 μm, 100 Å, Phenomenex, Torrance, United States), connected to an Ultra Performance Liquid Chromatography (UPLC) Acquity chromatography system (Waters, Manchester, United Kingdom). An ACN linear gradient containing 0.1% (v/v) formic acid (FA) is used (from 5 to 15% of ACN for 30 min, then from 15 to 30% of ACN for 60 min, from 30 to 50% of ACN for 10 min and finally from 50 to 95% of ACN for 10 min) with a flow rate of 500 μL·min⁻¹. The UPLC eluents are directly sprayed by electrospray at a voltage of 3 kV, the desolvation is carried out using dinitrogen (N2) at a flow rate of 600 L·h⁻¹ at a temperature of 300° C. The chromatographic system is coupled to a Synapt-G2-Si mass spectrometer (Waters, Manchester, United Kingdom). The MS measurements are made in data dependent mode (data dependent analysis, DDA) and the data are recovered in a mass range ranging from 100 to 2000 m/z. A maximum of 15 precursor ions with an intensity threshold of 10,000 are selected for fragmentation by the collision-induced dissociation (CID) method at a power of 8 to 9 V for low masses and a power of 40 to 90 V for high masses. The MS/MS spectra are recovered in a mass range ranging from 100 to 2000 m/z. The spectra are processed by Mass Lynx software (version 4.1.) (Waters, Manchester, United Kingdom). The analysis of the control sample (CTLF) allowed the base heterogeneity to be defined. 596 peptides originating from 22 milk proteins (αₛ₁-casein, β-casein, αₛ₂-casein, GLYCAM1, _{K}-casein, BTN1A1, β-lactoglobulin, FGFBP, lactoperoxidase, osteopontin, perilipin, α2 collagen, TRIPE, NPT2B, acylCoA desaturase, dysferlin, COG8, B4GT1, PSME4, DHX9, RALYL and apolipoprotein A2) were identified in this sample. 32% of them originate from β-casein. By comparison, between 150 and 722 peptides were identified in the samples fermented by the different tested strains. In these samples, the β-casein peptides represent 44 to 67% of all identified sequences. Due to the preponderance of β-casein peptides among the identified peptides, and the natural abundance of β-casein in milk, only peptides derived from β-casein will be studied. The peptides produced by the strains of the invention are therefore all derived from the proteolysis of β-casein in cow's milk. The data bank research is carried out using the Peaks Studio software (version 7.0.) (Bioinformatics Solutions, Waterloo, Canada) using the UniProt database (on May 15, 2017) restricted to the complete proteome of the Bos taurus species. The tolerance thresholds for the masses of the precursor ions and fragments are defined at ppm and 0.2 Da. The peptide sequences identified by the software are filtered according to a false positive rate (false discovery rate) strictly less than 1%.

For all strains, the peptides identified originate from the fermentate which has not undergone ultrafiltration. These same peptides should also be found in view of their mass in the ultrafiltered fermentate.

It should be noted that when identifying the peptides, the peptides which may originate from yeast have been considered. After analysis by mass spectrometry, the Applicant has observed, using the databases, that no peptide originating from the yeast was present in the fermentate which is ultrafiltered or not.

Table 4 groups the peptides which are non-phosphorylated and considered as novel, which are produced by the VF45A strain according to the fermentation method indicated above at pH 6.

Table 5 groups the peptides which are phosphorylated and considered as novel, which are produced by the VF45A strain according to the fermentation method indicated above at pH 6.

TABLE 4
VF 45A STRAIN

| SEQ ID NO | peptide |
|---|---|
| 1 | EEIVPN |
| 2 | SEEIVPN |
| 3 | SSEEIVPN |
| 4 | LTDVEN |
| 5 | SEEQQQTEDELQD |
| 6 | HNSLPQ |
| 7 | LPVPQKAVPYPQ |
| 8 | VLPVPQKAVPYPQ |
| 9 | DMPIQAF |
| 10 | DELQDKIHPF |
| 11 | EDELQDKIHPF |
| 12 | EEQQQTEDELQDKIHPF |
| 13 | EQQQTEDELQDKIHPF |
| 14 | QDKIHPF |
| 15 | QQQTEDELQDKIHPF |
| 16 | QQTEDELQDKIHPF |
| 17 | QTEDELQDKIHPF |
| 18 | SEEQQQTEDELQDKIHPF |
| 19 | TEDELQDKIHPF |

TABLE 5
VF 45A STRAIN

| SEQ ID NO | peptide |
|---|---|
| 20 | DIGSESTEDQAMEDIK |
| 21 | DIGSESTEDQAMEDIKQM |
| 22 | ESTEDQAMEDIKQM |
| 23 | ESTEDQAMEDIKQMEAE |
| 24 | FQSEEQQ |
| 25 | GSESTEDQAMEDIKQM |
| 26 | GSESTEDQAMEDIKQME |
| 27 | HNSLPQ |
| 28 | IGSESTEDQAMEDIKQME |
| 29 | IGSESTEDQAMEDIKQM |
| 30 | KDIGSESTEDQAMEDIKQM |
| 31 | SEEQQQTEDELQDKIHPF |
| 32 | SESTEDQAMEDIK |
| 33 | SESTEDQAMEDIKQM |
| 34 | SESTEDQAMEDIKQME |
| 35 | SESTEDQAMEDIKQMEAE |
| 36 | SSEEIVPN |
| 37 | STEDQAMEDIKQM |
| 38 | STEDQAMEDIKQME |
| 39 | STEDQAMEDIKQMEAE |
| 40 | SESTEDQAMEDIK |
| 41 | SESTEDQAMEDIKQM |
| 42 | SESTEDQAMEDIKQME |
| 43 | SESTEDQAMEDIKQMEAE |

Table 6 below groups the peptides which are non-phosphorylated and considered as novel, which are produced by the VFH049 strain according to the fermentation method indicated above at pH 6.

Table 7 groups the peptides which are phosphorylated and considered as novel, which are produced by the VFH049 strain according to the fermentation method indicated above at pH 6.

It is known that the phosphorylated peptides may be active in the calcium absorption (confers the publication entitled “Casein phosphopeptide promotion of calcium uptake in HT-29 cells—Relationship between biological activity and supramolecular structure” by Gravaghi, C., et al. published in 2007, FEBS J. 274, 4999-5011. doi: 10.1111/j.1742-4658.2007.06015.x.

TABLE 6
VF49d STRAIN

| SEQ ID NO | peptide |
|---|---|
| 44 | SLVYPFPGPIHNSLPQN |
| 45 | SLVYPFPGPIPNSLPQN |
| 46 | LEIVPN |
| 47 | KYKVPQLEIVPN |
| 48 | SLVYPFPGPIPN |
| 49 | FPKYPVEPFTESQ |
| 50 | PKYPVEPFTESQ |
| 51 | MPFPKYPVEPFTESQ |
| 52 | HKEMPFPKYPVEPFTESQ |
| 53 | EMPFPKYPVEPFTESQ |
| 54 | TLTDVEN |
| 55 | VYPFPGPIHN |
| 56 | SLVYPFPGPIHN |
| 57 | LVYPFPGPIHN |
| 58 | TPVVVPPFLQPEVMGVSKVKEAMAP |
| 59 | LPPITQ |
| 60 | PPITQ |
| 61 | NLPPITQ |
| 62 | VYPFPGPIHNSLPQ |
| 63 | KAVPYPQ |
| 64 | SLSQSKVLPVPQKAVPYPQ |
| 65 | YPFPGPIHNSLPQ |
| 66 | LVYPFPGPIHNSLPQ |
| 67 | SLVYPFPGPIHNSLPQ |
| 68 | TLTDVENL |
| 69 | TEDELQDKIHPF |
| 70 | SLVYPF |
| 71 | ELQDKIHPF |
| 72 | NIPPLTQTPVVVPPF |
| 73 | HKEMPFPKYPVEPF |
| 74 | DELQDKIHPF |

TABLE 7
VF49d STRAIN

| SEQ ID NO | peptide |
|---|---|
| 75 | ASPEVIE |
| 76 | EASPEVIE |
| 77 | EASPEVIESPPEINTVQ |
| 78 | KDIGSESTEDQAM |
| 79 | KFQSEEQQQTEDELQ |
| 80 | MESTEVF |
| 81 | SEEQQQTEDELQ |
| 82 | SEEQQQTEDELQDKIHPF |
| 83 | TLEASPE |
| 84 | TLEASPEVIESPPEINTVQ |
| 85 | TLEDSPEVIESPPEINTVQ |
| 86 | VPQLEIVPNSAEERLH |

Table 8 below groups the peptides which are non-phosphorylated and considered as novel, which are produced by the VF50b strain according to the fermentation method indicated above at pH 6.

Table 9 groups the peptides which are phosphorylated and considered as novel, which are produced by the VF50b strain according to the fermentation method indicated above at pH 6.

TABLE 8
VF50b STRAIN

| SEQ ID NO | peptide |
|---|---|
| 87 | SLVYPFPGPIPNSLPQN |
| 88 | SETLTDVENLHLPLPLLQSW |
| 89 | DVENLHLPLPLLQSW |
| 90 | VENLHLPLPLLQSW |
| 91 | NLHLPLPLLQSW |
| 92 | LHLPLPLLQSW |
| 93 | HLPLPLLQSW |
| 94 | AQTQSLVYPFPGPIPN |
| 95 | SLVYPFPGPIPN |
| 96 | VMGVSKVKEAMAPKNKEMPFPKYPVEPFTESQ |
| 97 | GVSKVKEAMAPKHKEMPFPKYPVEPFTESQ |
| 98 | KVKEAMAPKHKEMPFPKYPVEPFTESQ |
| 99 | EAMAPKHKEMPFPKYPVEPFTESQ |
| 100 | AMAPKHKEMPFPKYPVEPFTESQ |
| 101 | MAPKHKEMPFPKYPVEPFTESQ |
| 102 | APKHKEMPFPKYPVEPFTESQ |
| 103 | KHKEMPFPKYPVEPFTESQ |
| 104 | HKFMPFPKYPVEPFTESQ |
| 105 | KEMPFPKYPVEPFTESQ |
| 106 | MPFPKYPVEPFTESQ |
| 107 | PFPKYPVEPFTESQ |
| 108 | PKYPVEPFTESQ |
| 109 | PFTESQ |
| 110 | SVLSLSQ |
| 111 | TLTDVEN |
| 112 | QTEDELQD |
| 113 | TEDELQD |
| 114 | NIPPLTQTPVVVPP |
| 115 | DELQDKEHPFAQT |
| 116 | QTEDELQDKIHPFAQTQ |
| 117 | DELQDKHIPFAQTQ |
| 118 | ELQDKINPFAQTQ |
| 119 | QDKHIPFAQTQ |
| 120 | DKIHPFAQTQ |
| 121 | NLHLPLPLLQSWMHQPHQPLPFTVMFPPQ |
| 122 | LLQSWMHQPHQPLPPTVMFPPQ |
| 123 | LQSWMHQPHQPLPPTVMFPPQ |
| 124 | SWMHQPHQPLPPTVMFPPQ |
| 125 | WMHQPHQPLPPTVMFPPQ |
| 126 | MHQPHQPLPPTVMFPPQ |
| 127 | NQPHQPLPPTVMFPPQ |
| 128 | SVLSLSQSKVLPVPQ |
| 129 | SVLSLSQSKVLPVPQKAVPYPQ |
| 130 | SLSQSKVLPVPQKAVPYPQ |
| 131 | SLSQSKVLPVPQ |
| 132 | LSQSKVLPVPQKAVPYPQ |
| 133 | LSQSKVLPVPQ |
| 134 | SQSKVLPVPQKAVPYPQ |
| 135 | SQSKVLPVPQ |
| 136 | SKVLPVPQKAVPYPQ |
| 137 | VLPVPQKAVPYPQ |
| 138 | KAVPYPQ |
| 139 | TDVENL |
| 140 | SVLSLSQSKVIPVPQKAVPYPQRDMPIQAF |
| 141 | SLSQSKVLPVPQKAVPYPQRDMPIQAF |
| 142 | SQSKVLFVFQKAVPYPQRDMPIQAF |
| 143 | SKVLPVPQKAVPYPQRDMPIQAF |
| 144 | VIPVPQKAVPYPQRDMPIQAF |
| 145 | KAVPYPQRDMPIQAF |
| 146 | VPYPQRDMPIQAF |
| 147 | EQQQTEDELQDKIHPF |
| 148 | QQQEEDELQDKIHPF |
| 149 | DELQDKIHPF |
| 150 | LQDKIHPF |
| 151 | SLPQNIPPLTQTPVVVPPF |
| 152 | NIPPLTQTPVVVPPF |
| 153 | IPPLTQTPVVVPPF |
| 154 | VMGVSLVKEAMAPKHKEMPFPKYPVEPF |
| 155 | EAMAPKHKEMPFPKYPVEPF |
| 156 | MAPKHKLMPFPKYPVEPF |
| 157 | APKNKFMPFPLYPVFPF |
| 158 | HKEMPTPKYPVEPF |
| 159 | KEMPFPKYPVEPF |
| 160 | EMPFPKYPVEPF |
| 161 | MPFPKYPVEPF |

TABLE 9
VF 50b STRAIN

| SEQ ID NO | peptide |
|---|---|
| 162 | SVEQKHIQ |
| 163 | SVEQKHIQK |
| 164 | MESTEVFTK |
| 165 | MESTEVFTKK |
| 166 | SAEERLHSM |
| 167 | DIGSESTE |
| 168 | DLISKEQIVIR |
| 169 | EKFQSEE |
| 170 | GKEKVNELSKD |
| 171 | ESKEQIVIR |
| 172 | KDIGSESTE |
| 173 | KDIGSESTEDQAME |
| 174 | KFQSEEQQQTED |
| 175 | KFQSEEQQQTEDEL |
| 176 | KIEKFQSEE |
| 177 | KKIEKFQSE |
| 178 | KKIEKFQSEE |
| 179 | KVPQLEIVPNSAEERLH |
| 180 | LISKEQIVIR |
| 181 | MESTEVFTK |
| 182 | MESTEVFTKK |
| 183 | NREQLSTSEENSKKTVD |
| 184 | SAEERLHS |
| 185 | SAEERLHSMK |
| 186 | SAEERLHSMKEG |
| 187 | SAEERLHSMKEGIH |
| 188 | SEEQQQTEDELQ |
| 189 | SEEQQQTEDELQD |
| 190 | SKDIGSESTE |
| 191 | SKDIGSESTEDQAM |
| 192 | SKDIGSESTEDQAME |
| 193 | SKEQIVIR |
| 194 | STEVFTK |
| 195 | STSEENSKKTVD |
| 196 | SVEQKIH |
| 197 | SVEQKHIQ |
| 198 | SVEQKHIQKE |
| 199 | VPQLEIVPNSAEERLH |

### Biological Activities of the Fermentates and the Peptides

Fermentates (before and after ultrafiltration) were tested for two biological activities, namely ACE inhibition and the ability to modulate THE calcium absorption. Moreover, the toxicity of the hydrolysates was assessed. For analyzes, the various samples are diluted in H₂O at a concentration of 15 g/L of dry matter.

### Cytotoxicity Test

The aim of this experiment is to test the possible toxicity of the fermentates towards the intestinal cells. The cell viability is measured by the use of the CCK-8 reagent (Cell Counting Kit-8), this method is based on the reduction of a tetrazolium salt by cellular dehydrogenases producing formazan whose formation is followed at 450 nm. The Caco-2 cells are seeded in 96 well plate at a density of 8,000 cells per well in a volume of 200 μl of complete DMEM medium, and cultured for 7 days at 37° C., 5% of CO₂. The fermentates are diluted in the complete DMEM medium at a concentration of 5 and 10 g/L. A PBS buffer also diluted in the DMEM medium (same dilution factor as the samples) is used as a control. A volume of 150 μL of sample is deposited in each well, followed by an incubation for 7 or 24 h at 37° C., 5% of CO₂. After incubation, the Caco-2 cells are washed twice with the PBS buffer then a volume of 150 μL of complete DMEM medium supplemented with 5% (v/v) of CCK-8 reagent (Sigma-Aldrich, St Louis, United States) is added into each well. The plate is then incubated for 2 h at 37° C., 5% of CO₂ in the dark, then read at 450 nm with the Xenius XC spectrofluorimeter (Safas Monaco, Monaco, France). The cell viability is calculated relative to the absorbance obtained for the control wells (corresponding to 100% of viable cells). At a concentration of 5 g/L, the crude fermentates (unfiltered) of each of the strains of the invention have no toxicity towards the Caco-2 cells even after 24 hours of contact. The same observation is made for a concentration of 10 g/L of dry matter. The ultrafiltered fermentates of each of the strains do not have a cytotoxicity either.

### Inhibition of the Angiotensin Converting Enzyme

The aim is to study the inhibitory potential of the fermentates towards ACE. It is based on the use of a fluorescent substrate, o-aminobenzoyl-Gly-p-nitro-L-Phe-Pro (Abz-GIy-Phe(NO₂)-Pro). The hydrolysis of this substrate by ACE generates the Abz-GIy fluorophore group which can be followed by excitation and emission wavelengths of 355-375 nm and 400-430 nm respectively. The samples, enzyme and substrate are prepared and diluted in a Tris-HCl buffer (150 mM) at pH 8.3 at the desired concentrations. The hydrolysis reaction is carried out in a 96 well plate in a final volume of 300 μL comprising 50 μL of sample or of Tris-HCl buffer (for the negative inhibition indicator), 50 μL of an ACE solution (0.05 U/mL) (Sigma-Aldrich, St Louis, United States) and 200 μL of Abz-GIy-Phe (NO₂)-Pro (0.45 mM) substrate (Bachem, Bubendorf, Switzerland). An indicator containing no enzyme (replaced by the Tris-HCl buffer) is also carried out. The plate is then incubated at 37° C. for 1 hour, a fluorescence measurement takes place every 2 min with excitation and emission wavelengths of 365 and 415 nm respectively by a Xenius XC spectrofluorimeter (Safas Monaco, Monaco, France) equipped with a water bath at 37° C. The inhibition percentages and the IC₅₀ of the different tested samples are calculated depending on the negative inhibition indicator.

For the analysis of the biological activities (of all the biological activities tested in the present application), the significance of the results is assessed by a one-factor analysis of variance (ANOVA) followed by a Tukey test for the multiple comparison of the means. The difference between the means is considered as significant for a p value<0.05. The statistical tests were carried out using the R software (R core team, 2016, Vienna, Austria), on the R Commander package.

In order to demonstrate the interest of the fermentation in terms of the studied biological activities, the activities are compared with those of the control fermentate (CTLF), fermentation medium incubated without inoculation.

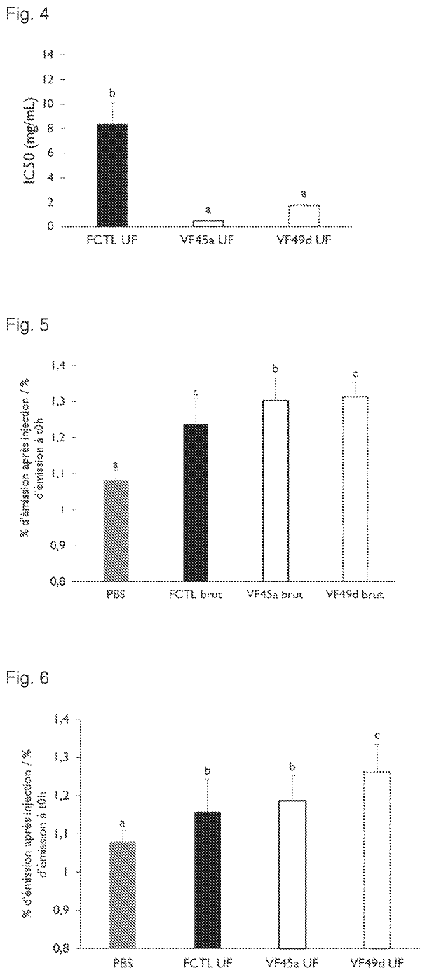

The samples were tested at different concentrations of dry matter, this is in order to be able to calculate the concentration inhibiting half of the enzymatic activity (IC₅₀). The results are represented in FIGS. 3 and 4.

The crude fermentates have IC₅₀ ranging from 12.76 mg/mL for the crude control fermentate at 0.56 mg/mL for the fermentate produced by the VF45A strain. For the VFH049 strain, the IC₅₀ of the crude fermentate is 0.76 mg/mL. For the ultrafiltered fermentates, the IC₅₀ are 8 mg/mL for the control, 0.47 mg/mL for the fermentate produced by the VF45A strain and 1.76 mg/mL for the fermentate produced by the VFH049 strain. The VF45A and VFH049 strains very significantly improve the capacity of the fermentates to inhibit ACE relative to the control. The same effect is obtained with the ultrafiltered fermentates. Molecules of interest are therefore produced by the strains during the fermentation and have an ACE inhibitory activity.

Prediction of the ACE Inhibitory Activity by QSAR Model; Comparison with the Peptides of the Prior Art

In order to predict the ACE inhibitory activity of each of the identified peptides (non-phosphorylated peptides), a QSAR (Quantitative Structure Activity Relationship) statistical model was used. This model was developed by Pripp et al. (2004), using the z-scores (z1, z2 and z3) as amino acid descriptors, described by Hellberg et al. (1987). The prediction is based on the physico-chemical characteristics of the last two amino acids of the peptides (in the C terminal position) allowing obtaining a predictive IC₅₀ value via the equation of the model. The QSAR approach used to determine the IC₅₀ of our peptides was used on the VPP and IPP peptides of the prior art. Their IC₅₀ is 26 μM, while some of the peptides of the invention have an IC₅₀ value below 20 μM (up to 8.7 μM for the most active), and are therefore a priori more effective.

The results are collected in Tables 10 to 12 below.

TABLE 10
VF 45A STRAIN

| SEQ ID NO | Log IC50 | IC50 (μM) |
|---|---|---|
| 1 | 1.2729 | 18.7456282 |
| 2 | 1.2729 | 18.7456282 |
| 3 | 1.2729 | 18.7456282 |
| 4 | 1.3041 | 20.1418798 |
| 5 | 1.4138 | 25.9298497 |
| 6 | 1.5225 | 33.3042762 |
| 7 | 1.5225 | 33.3042762 |
| 8 | 1.5225 | 33.3042762 |
| 9 | 1.6101 | 40.7474091 |
| 10 | 1.6498 | 44.6477934 |
| 11 | 1.6498 | 44.6477934 |
| 12 | 1.6498 | 44.6477934 |
| 13 | 1.6498 | 44.6477934 |
| 14 | 1.6498 | 44 6477934 |
| 15 | 1.6498 | 44.6477934 |
| 16 | 1.6498 | 44.6477934 |
| 17 | 1.6498 | 44.6477934 |
| 18 | 1.6498 | 44 6477934 |
| 19 | 1.6498 | 44.6477934 |

TABLE 11
VF 49d STRAIN

| SEQ ID NO | Log IC50 | IC50 (μM) |
|---|---|---|
| 44 | 0.9406 | 8.72167702 |
| 45 | 0.9406 | 8.72167702 |
| 46 | 1.2729 | 18.7456282 |
| 47 | 1.2729 | 18.7456282 |
| 48 | 1.2729 | 18.7456282 |
| 49 | 1.2809 | 19.0941355 |
| 50 | 1.2809 | 19.0941355 |
| 51 | 1.2809 | 19.0941355 |
| 52 | 1.2809 | 19.0941355 |
| 53 | 1.2809 | 19.0941355 |
| 54 | 1.3041 | 20.1418798 |
| 55 | 1.3124 | 20.5305224 |
| 56 | 1.3124 | 20.5305224 |
| 57 | 1.3124 | 20.3305224 |
| 58 | 1.3893 | 24.5075538 |
| 59 | 1.5164 | 32.8397619 |
| 60 | 1.5164 | 32.8397619 |
| 61 | 1.5164 | 32.8397619 |
| 62 | 1.5225 | 33.3042762 |
| 63 | 1.5225 | 33.3042762 |
| 64 | 1.5225 | 33.3042762 |
| 65 | 1.5225 | 33.3042762 |
| 66 | 1.5225 | 33.3042762 |
| 67 | 1.5225 | 33.3042762 |
| 68 | 1.5249 | 33.4888319 |
| 69 | 1.6498 | 44.6477934 |
| 70 | 1.6498 | 44.6477934 |
| 71 | 1.6498 | 44.6477934 |
| 72 | 1.6498 | 44.6477934 |
| 73 | 1.6498 | 44.6477934 |
| 74 | 1.6498 | 44.6477934 |

TABLE 12
VF 50b STRAIN

| SEQ ID NO | Log IC50 | IC50 (μM) |
|---|---|---|
| 87 | 0.9406 | 8.72167702 |
| 88 | 1.0943 | 12.4251031 |
| 89 | 1.0943 | 12.4251031 |
| 90 | 1.0943 | 12.4251031 |
| 91 | 1.0943 | 12.4251031 |
| 92 | 1.0943 | 12.4251031 |
| 93 | 1.0943 | 12.4251031 |
| 94 | 1.2729 | 18.7456782 |
| 95 | 1.2729 | 18.7456782 |
| 96 | 1.2809 | 19.0941355 |
| 97 | 1.2809 | 19.0941355 |
| 98 | 1.2809 | 19.0941355 |
| 99 | 1.2809 | 19.0941355 |
| 100 | 1.2809 | 19.0001355 |
| 101 | 1.2809 | 19.0941355 |
| 102 | 1.2809 | 19.0941355 |
| 103 | 1.2809 | 19.0941355 |
| 104 | 1.2809 | 19.0941355 |
| 105 | 1.2809 | 19.0941355 |
| 106 | 1.2809 | 19.0941355 |
| 107 | 1.2809 | 19.0941355 |
| 108 | 1.2809 | 19.0941355 |
| 109 | 1.2809 | 19.0941355 |
| 110 | 1.2809 | 19.0941355 |
| 111 | 1.3041 | 20.1418798 |
| 112 | 1.4138 | 25.9298497 |
| 113 | 1.4138 | 25.9298497 |
| 114 | 1.429 | 26.8534445 |
| 115 | 1.4648 | 29.160838 |
| 116 | 1.5164 | 32.8397619 |
| 117 | 1.5164 | 32.8397619 |
| 118 | 1.5164 | 32.8397619 |
| 119 | 1.5164 | 32.8397619 |
| 120 | 1.5164 | 32.8397619 |
| 121 | 1.5225 | 33.3042762 |
| 122 | 1.5225 | 33.3042762 |
| 123 | 1.5225 | 33.3042762 |
| 124 | 1.5225 | 33.3042762 |
| 125 | 1.5225 | 33.3042762 |
| 126 | 1.5225 | 33.3042762 |
| 127 | 1.5225 | 33.3042762 |
| 128 | 1.5225 | 33.3042762 |
| 129 | 1.5225 | 33.3042762 |
| 130 | 1.5225 | 33.3042762 |
| 131 | 1.5225 | 33.3042762 |
| 132 | 1.5225 | 33.3042762 |
| 133 | 1.5225 | 33.3042762 |
| 134 | 1.5225 | 33.3042762 |
| 135 | 1.5225 | 33.3042762 |
| 136 | 1.5225 | 33.3042762 |
| 137 | 1.5225 | 33.3042762 |
| 138 | 1.5225 | 33.3042762 |
| 139 | 1.5249 | 33.4888319 |
| 140 | 1.6101 | 40.7474091 |
| 141 | 1.6101 | 40.7474091 |
| 142 | 1.6101 | 40.7474091 |
| 143 | 1.6101 | 40.7474091 |
| 144 | 1.6101 | 40.7474091 |
| 145 | 1.6101 | 40.7474091 |
| 146 | 1.6101 | 40.7474091 |
| 147 | 1.6498 | 44.6477934 |
| 148 | 1.6498 | 44.6477934 |
| 149 | 1.6498 | 44.6477934 |
| 150 | 1.6498 | 44.6477934 |
| 151 | 1.6498 | 44.6477934 |
| 152 | 1.6498 | 44.6477934 |
| 153 | 1.6498 | 44.6477934 |
| 154 | 1.6498 | 44.6477934 |
| 155 | 1.6498 | 44.6477934 |
| 156 | 1.6498 | 44.6477934 |
| 157 | 1.6498 | 44.6477934 |
| 158 | 1.6498 | 44.6477934 |
| 159 | 1.6498 | 44.6477934 |
| 160 | 1.6498 | 44.6477934 |
| 161 | 1.6498 | 44.6477934 |

The QSAR approach used to determine the IC₅₀ of the peptides of the invention was used on the VPP and IPP peptides described in the prior art. Their IC₅₀ is 26 μM, while some of our peptides are below 20 μM (up to 8.7 μM for the most active), and are therefore a priori more effective.

### Modulation of the Calcium Absorption

The majority of the consumed calcium is absorbed in the intestines, two calcium transport pathways has been identified in the intestine, the paracellular pathway and the transcellular pathway. The paracellular transport of calcium is a passive diffusion of this element from the lumen to the intestinal mucosa, according to the gradient formed between these two compartments. Two proteins are involved in this transport, claudins 2 and 12 (CLD-2-12) which are part of the tight junctions between the cells and acting as calcium channels (Fujita et al., 2008). The transcellular calcium transport is an active transport involving the incorporation of calcium within intestinal cells by a transporter called TRPV6. Calcium is subsequently transported and excreted at the basal pole of the cell in the intestinal mucosa. These two calcium transport pathways are regulated by vitamin D, a hormone which, once fixed at its nuclear receptor the VDR (Vitamin D Receptor) acts as a transcription factor controlling the genes of CLD-2-12 and TRPV6.

### On Caco-2 Cells, Measurement of Calcium Incorporation

The ability of the fermentates to modulate the calcium absorption was assessed using the Caco-2 cells. The test consists contacting the fermentates (concentration of 10 g/L) with the Caco-2 cells for 7 h. After contact, the incorporation of calcium by the cells is assessed by using an intracellular probe capable of emitting a fluorescence in the presence of calcium. After rinsing the Caco-2 cells, the FluoForte® probe is added following the instructions of the FluoForte® calcium assay kit (Enzo Life Sciences, Farmingdale, United States), the plate is then incubated at ambient temperature for 1 hour, time of penetration of the probe. The fluorescence emission is then followed by a Xenius XC spectrofluorimeter (Safas Monaco, Monaco, France) with an excitation wavelength at 490 nm for an emission followed at 525 nm. The emission is measured for 30 sec with an injection of 25 μL of a 250 mM CaCl₂ solution between 9 and 10 sec of kinetics by means of an injector module coupled to the spectrofluorimeter. This injection causes a sudden entry of calcium within the intestinal cells which leads to an increase in the fluorescence emission. The ability of bacterial strains to modulate the incorporation of calcium in the cells is determined by this increase in fluorescence after calcium injection. The results are thus expressed for each well relative to the fluorescence mean measured between 0 and 9 sec of kinetics, considered as the basal fluorescence of a well. A fluorescence increase ratio, for the different contact conditions, is therefore obtained.

The effect of the fermentates obtained with the VF45A and VFH049 strains is compared with that of the PBS buffer and that of the control fermentate. The results show that the crude fermentates obtained with these two strains are capable of positively modulating the incorporation of calcium relative to the PBS buffer and to the control (FIG. 5). Thus, the ratio of fluorescence emission to basal emission is 1.08 for the PBS, 1.23 for the control fermentate, 1.3 for the fermentate produced by the VF45A strain and 1.31 for the fermentate produced by the VFH049 strain. When the ultrafiltered fermentates are tested, however the effect is less pronounced for the VF45a strain than for the VF49d strain (FIG. 6). Nevertheless, a tendency to increase absorption is observed for both strains.

Modulation of the Expression of the trpv6 Gene in Caco-2 Cells

The objective of this part is to assess the ability of fermentates to modulate the expression of the trpv6 gene by the intestinal cells. An RT-qPCR approach is conducted using the Caco-2 cells as a model. The Caco-2 cells are seeded in a 24 well plate at a density of 40,000 cells per well in a final volume of 500 μL of complete DMEM medium and incubated for 15 days at 37° C., 5% CO₂ with a change of the medium every two days after 7 days of culture. The fermentates are diluted in complete DMEM medium at a concentration of 10 g/L of dry matter. The PBS buffer diluted in the DMEM medium (same dilution factor as for the samples) is used as a control. After incubation, the cells are washed twice with PBS buffer, then a volume of 300 μL of each sample is deposited in the wells. The plate is then incubated for 7 h at 37° C., 5% CO₂. After contact between the Caco-2 cells and the samples, the wells are rinsed twice with the PBS buffer then an extraction of the RNAs with the TRIzol™ reagent is carried out. The RNA samples are first treated with DNase to eliminate the possible DNA fragments co-extracted and/or remaining following the extraction. An 8 μL volume containing 1000 ng of RNA is mixed with 1 μL of DNase, and 1 μL of DNase buffer (ThermoScientific, Waltham, United States). The reaction takes place at 37° C. for 30 min, it is stopped by adding 1 μL of 50 mM EDTA solution (ThermoScientific, Waltham, United States) followed by an incubation at 65° C. for 10 minutes.

Subsequently, the samples were retrotranscribed into cDNA using the RevertAid H minus first strand cDNA synthesis kit (ThermoScientific, Waltham, United States), according to the provided instructions. For the qPCR reaction, the cDNA samples are diluted (1/16) in H₂O. For a 2 μL volume of sample, 18 μL of qPCR mixture are added containing 10 μL of Power SYBR® Green PCR Master Mix (2×) (Applied Biosystems, Life Technologies, Foster City, United States), 0.6 μL of each primer (10 μM) and 6.8 μL of H₂O. the fluorescence is followed during the reaction in a CFX Connect Real Time Detection System thermocycler (Bio-Rad, Hercules, United States). After a denaturation step at 95° C. for 10 min, 40 cycles of PCRs are carried out successively, one cycle comprises denaturation at 95° C. for sec, a hybridization of 58 or 60° C. depending on the pair of primers used for 30 sec and an elongation at 72° C. for 30 sec. The realization of a melting curve ends the reaction.

The studied gene is the gene encoding the calcium transporter (transient receptor potential selective for calcium) (trpv6) also called CaT1 (calcium transporter 1). The expression of this gene is normalized relative to that of the gene encoding the peptidylprolyl isomerase A (ppiA). The used pairs of primers, as well as their hybridization temperatures, are presented below in Table 13 below:

TABLE 13

| | | Size of the | | ¤ |
|---|---|---|---|---|
| Gene¤ | Primers (5′-3′)¤ | amplicon (pb)¤ | Tm (° C.)¤ | |
| trpv6¤ | sens: TGATGCGGCTCATCAGTGCCAGC | 251¤ | 58¤ | ¤ |
| | (SEQ ID NO 200)¶ | | | |
| | antisens: GTAGAAGTGGCCTAGCTCCTCG | | | |
| | (SEQ ID NO 201)¤ | | | |
| ppia¤ | sens: TGCTGACTGTGGACAACTCG | 136¤ | 60¤ | ¤ |
| | (SEQ ID NO 202)¶ | | | |
| | antisens: TGCAGCGAGAGCACAAAGAT | | | |
| | (SEQ ID NO 203)¤ | | | |

The fermentate samples were contacted with the Caco-2 cells and the variations in the expression of the trpv6 gene were then studied by qPCR.

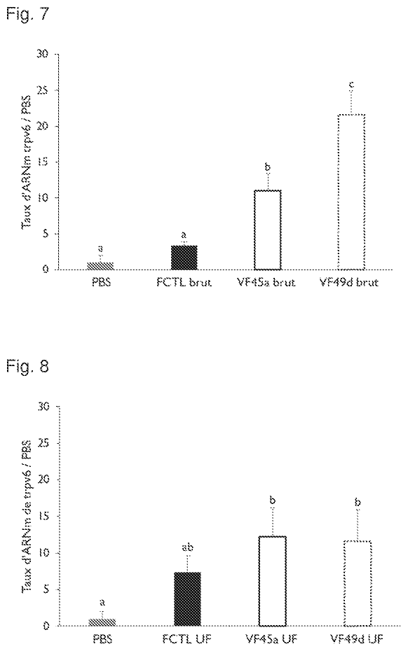

The fermentates obtained with the VF45A and VFH049 strains are capable of positively modulating the expression of the gene. More particularly, the fermentate produced by strain VFH049 causes an overexpression of the gene 20 times greater than the control (FIG. 7). The effect of the ultrafiltered fermentates is less pronounced, not significant even if a tendency to induce the gene is clearly visible for the two strains (FIG. 8). The fermentates produced by the two strains are therefore capable of modulating at the same time the calcium incorporation and the expression of trpv6 relative to the unfermented milk.

### Study of the Probiotic Characters

The significance of the results is assessed by a one-factor analysis of variance (ANOVA) followed by a Tukey test for the multiple comparison of the means. Concerning the calcium transport experiment through a membrane of Caco-2 cells, the post-ANOVA test is a Dunn test leading to a comparison to the mean of the control condition. The difference between the means is considered as significant for a p value<0.05. The statistical tests were carried out using the R software (R core team, 2016, Vienna, Austria), on the R Commander package.

### Acidity Tolerance Test

The principle of this test is based on the comparison of the survival of a bacterial strain between an incubation of 2 h at pH 2 and an incubation at the pH of the MRS medium (pH 6.2). For this experiment, each strain of the collection was cultured in liquid MRS medium for 24 h at 37° C. in anaerobic condition. A volume of 200 μL of culture was then diluted (1/10) either in the same MRS medium at pH 6.2 (called tube A), or in MRS medium adjusted to pH 2 with hydrochloric acid (HCl) (called tube B). The tubes A and B were incubated at 37° C. for 2 h then 10 μL of each tube were spread on MRS agar. The seeded dishes were incubated for 48 hours at 37° C. in anaerobic condition.

The acidity tolerance was assessed by comparing the numbers of colonies between the dishes A and B. The strains were divided into different acidity tolerance classes according to the result of the B/A ratio. Class 0 groups the strains which are not tolerant to acidity (no colony on dish B). Class 1 corresponds to the weakly tolerant strains (dish B=0 to 30% of the colonies on dish A). Class 2 groups strains having a moderate acidity tolerance (dish B=30 to 80% of the colonies on dish A). Finally, class 3 corresponds to the acidity tolerant strains (dish B has more than 80% of the colonies of dish A).

The strains were divided into 4 tolerance classes ranging from class 0 (non-tolerant strains) to class 3 (tolerant strains).

### Determination of the Parietal Hydrophobicity of the Strains

The objective of this test is to assess the hydrophobic character of the wall of the different strains. A simple test has already been developed to assess this criterion, it is the MATS (Microbial Adhesion To Solvents) test. It consists in measuring the affection of a bacterial strain for an apolar hydrocarbon (most often n-hexadecane) in order to estimate the hydrophobicity of the wall. For the experiment, the strains of the collection were cultured in liquid MRS medium for 24 hours at 37° C. in anaerobic condition. The cells are then washed twice by successively repeating a centrifugation step at 10,000 rpm for 10 min, followed by a step of resuspending the cells in a Phosphate Buffered Saline (PBS) buffer, pH 7.4. The Optical Density (OD at 630 nm (initial OD_{630 nm}) was determined with the ELx808 spectrophotometer (BioTek Instruments Inc., Vermont, United States). A volume of 1 mL of suspension was then mixed with 100 μL of n-hexadecane (Acros Organics, Geel, Belgium) by creating a vortex in the mixture for 1 min. The solution was then set to rest at ambient temperature for 15 min then 100 μL of the aqueous phase were collected for a new determination of the OD at 630 nm (final OD_{630 nm}). The parietal hydrophobicity of the strains was calculated according to the following formula:

$$\% H = ( \frac{initial {OD}_{630 nm} - final {OD}_{630 nm}}{initial {OD}_{630 nm}} ) \times 100 ¶ \qquad [ Math . 1 ]$$

With % H: the percentage of parietal hydrophobicity, initial OD_{630 nm}: the initial OD of the suspension and final OD_{630 nm}: the OD measured after addition and mixture of the suspension with n-hexadecane.

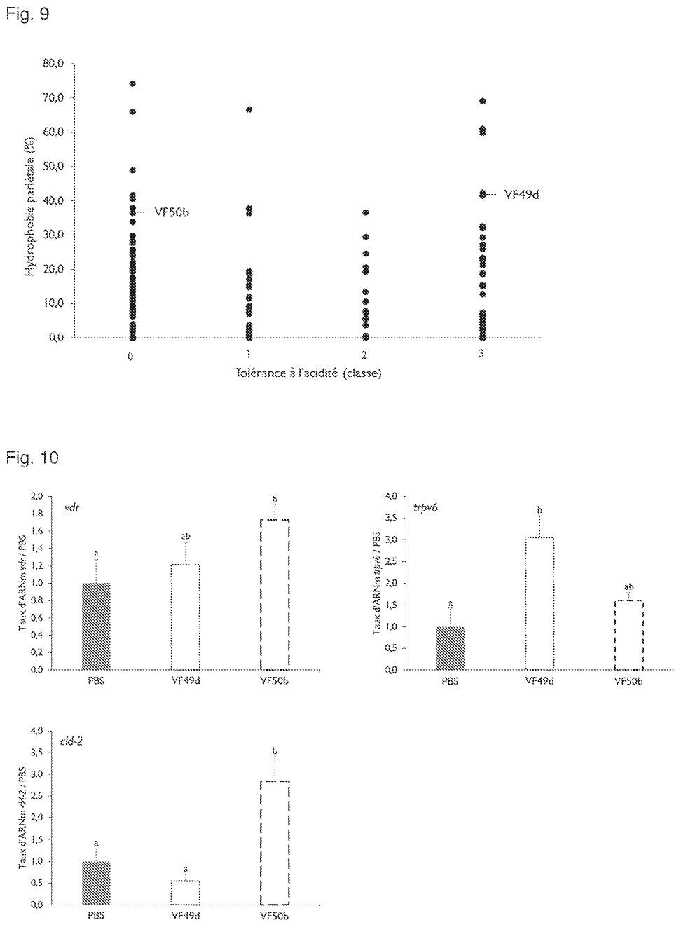

The results of the acidity resistance and parietal hydrophobicity tests are represented in FIG. 9 which represents the parietal hydrophobicity percentages obtained for the strains tested depending on their acidity tolerance class. In FIG. 9, it is noted that the VF50b strain does not withstand acidity, which is not the case with the VFH049 strain.

### Self-Aggregation Test

This test allows assessing the ability of the strains to be aggregated therebetween in a liquid medium. It is based on the comparison of the bacterial concentration above a suspension at time 0 with the bacterial concentration of the same suspension incubated for a given time without any agitation. The strains capable of a strong self-aggregation will aggregate with each other, thus increasing their sedimentation rate. The strains are cultured in MRS for 24 h at 37° C. then washed and resuspended in PBS buffer at an OD at 600 nm of 1. A volume of 4 mL of suspension is stirred so as to form a vortex for 10 sec then incubated at ambient temperature without stirring. After 2.5 and 5 hours of incubation, 100 μL of medium are carefully taken from the surface of the suspension. The taken samples are then diluted (1/10) in PBS buffer for a measurement of the OD at 600 nm. The self-aggregation percentage of the strains (% A) is then calculated according to the relationship:

$$\% A = ( 1 - \frac{{A}_{t}}{{A}_{0}} ) \times 100 ¶ \qquad [ Math . 2 ]$$

With A0: the initial absorbance at 600 nm of the suspension, measured at the start of the experiment (and equal to 1) and At: the absorbance at 600 nm measured at 2.5 or 5 h of incubation. The % of aggregation of the VFH049 strain is 5.38%±5.24 after 2.5 hours and 67.44%±6.99 after 5 hours. For the VF50b strain, the % of aggregation is 33.33%±14.8 after 2.5 hours and 66.67%±17.97 after 5 hours. The strains are therefore capable of adhering to the intestinal cells.

### Assessment of Tolerance to the Gastrointestinal Digestion

In order to assess the survival of the strains during and after their passage through the digestive tract, a static digestion model was used (Belguesmia el al. 2016). This model simulates the 3 main compartments of the digestive tract namely the mouth, stomach and intestine in a sequential manner. Each compartment is simulated by the addition of a fluid mimicking the physiological conditions of this step. All digestion is carried out in sterile condition, all fluids are autoclaved at 121° C. for 20 min before the experiment, the addition of the enzymes takes place after sterilization and is followed by sterilizing filtration (at 0.2 μm). The strains are cultured in an MRS medium for 24 h at 37° C. The bacterial cells are washed twice then resuspended in a PBS buffer at a concentration of 10⁹ CFU/mL. For the digestion, the buccal phase is simulated by adding 8 mL of PBS, adjusted to pH 6.8, to 1 ml of the bacterial suspension. The mixture is incubated under constant agitation at 200 rpm for 5 min at 37° C. The gastric phase is simulated by adding 12 mL of PBS at pH 3 supplemented with bovine pepsin (Sigma-Aldrich, St Louis, United States) at 1.56 mg/mL. The suspension is incubated for 2 hours at 37° C. with stirring at 200 rpm. During the incubation, the pH of the solution is maintained between 3 and 3.5 by the addition of hydrochloric acid (HCl) (1 M) or sodium hydroxide (NaOH) (1 M). For the intestinal phase, 1 ml of 1 M sodium carbonate (NaHCO₃) is added to the mixture in order to raise the pH to about 7 and inactivate the pepsin. A volume of 12 mL of PBS at pH 8.2 supplemented with bovine pancreatic enzymes (Sigma-Aldrich, St Louis, United States) at 0.28 mg/mL, then a volume of 6 mL of PBS at pH 8.1 containing 60 g/L of Ox-bile (Sigma-Aldrich, St Louis, United States) are added to the mixture in order to simulate the intestinal conditions. This step takes place for 2 h at 37° C. with stirring at 200 rpm while maintaining the pH between 7 and 7.5. At the end of each step of the digestion, a sample of 100 μL of the reaction mixture is taken, thus for the same digestion, 4 samples are obtained comprising the sampling of the stock tube (TM), of the salivary phase (S), of the gastric phase after 2 h of incubation (G2), and finally that after 2 hours of intestinal phase (I2). Each sample is then diluted (1/10) successively in a PBS buffer. For the determination of the bacterial concentration in the samples, 100 μL of the appropriate dilutions are spread on MRS agar. The seeded plates are then incubated at 37° C. for 48 hours in anaerobic condition. After incubation, the enumeration of the CFUs allows determining the bacterial concentration in the samples. The results are expressed in CFU/mL. The initial number of viable bacterial cells is 10⁹ CFU/mL in the salivary phase. During DGI, the amount of bacteria decreases more or less according to the strain, the gastric phase being the most deleterious for all strains. At the end of this phase, the VFH049 strain is present in an amount of 10^{7.8} CFU/mL while the VF50b strain is present in an amount equal to 10^{7.2} CFU/mL. The VFH049 and VF50b strains therefore tolerate the gastrointestinal digestion.

### Cytotoxicity Test

The objective of this test is to assess the possible toxicity of the strains relative to the intestinal barrier. The Caco-2 and HT-29 MTX cells are used for this purpose.

For the cytotoxicity test, the cells are seeded in a 96 well plate at a density of 8,000 cells per well in a volume of 150 μL of medium and cultured for 7 days at 37° C., 5% of CO₂. Before adding the bacterial strains, the intestinal cells are washed twice with PBS buffer. The bacterial strains, for their part, are cultured as previously described and suspended in the DMEM medium without addition at a concentration of 10⁷ CFU/mL. For each well, 150 μL of bacterial suspension are added. PBS buffer, diluted with DMEM without addition, as for the strains, is used as a negative control. The contact between the bacterial strains and the intestinal cells takes place for 24 h at 37° C., 5% of CO₂. The strains are contacted with both Caco-2 cells and HT-29 MTX cells independently. The determination of the intestinal cell mortality is used herein to assess the possible toxicity of the bacterial strains. The mortality of the cells is assessed by assaying the activity of lactate dehydrogenase (LDH). This activity is assayed by the LDH activity assay kit (Sigma-Aldrich, St Louis, United States), this kit is based on the reduction reaction of oxidized Nicotinamide Adenine Dinucleotide (NAD) in its reduced form (NADH) by lactate dehydrogenase, NADH can be detected at 450 nm. After contact, 50 μL of supernatant are taken from each well and the LDH activity is assayed according to the kit protocol. The absorbance of the plate is read at 450 nm by a Xenius XC spectrofluorimeter (Safas Monaco, Monaco, France). The intestinal cell mortality is calculated as a percentage of the mean of the absorbances of the control condition (the proportion of LDH released in this condition is taken as a reference at 100%). For VFH049 and VF50b strains, the results show an absence of significant toxicity towards the two cell lines, which suggests that under these experimental conditions, the selected bacterial strains are not deleterious for the intestinal cells.

### Assessment of the Adhesion of the Strains to Intestinal Cells

The objective of this test is to assess the ability of the strains to adhere to intestinal cells. It is based on the comparison between the amount of bacterial cells adhering to the Caco-2 cell monolayer relative to the initially added amount of bacteria. The strains are cultured and prepared as previously described, a suspension in DMEM medium without addition is prepared at a concentration of 10⁷ CFU/mL. The Caco-2 cells are seeded in 24 well plates at a concentration of 40,000 cells per well in a volume of 500 μL of complete DMEM. After incubation for 7 days at 37° C., 5% of CO₂, the cells are washed twice with a PBS buffer. A volume of 300 μL of bacterial suspension is added into each well, the plate is then incubated at 37° C., 5% of CO₂, for 2 hours. The used bacterial suspension is stored for determining the CFU concentration. After 2 hours of contact, the Caco-2 cells are washed twice with a PBS buffer in order to remove the non-adherent bacteria. The intestinal cells are then lysed by adding 100 μL of PBS buffer supplemented with 0.1% (v/v) Triton X-100. After incubation for 15 min at ambient temperature, the lysate and the stock suspension used are successively diluted (1/10) in PBS, then spread on MRS agar. The seeded plates are incubated for 48 hours at 37° C. in anaerobic condition. After enumeration and determination of the bacterial concentration in CFU/mL, the percentage of adherent bacterial cells is calculated relative to the concentration obtained in the stock suspension representing the amount of bacteria added at the start of the experiment (set at 100%). The % of cells adhering to Caco-2 cells is 1.48±0.41 for the VFH049 strain and 0.18±0.13 for the VF50b strain. The VFH049 strain is much more adherent to intestinal cells which tends to indicate that it is more likely to have a probiotic action.

### Biological Activities of the Strains on the Calcium Absorption

### General Contact Protocol Between Bacterial Strains and Intestinal Cells

This contact protocol is used for each technique employed in this part. The bacterial strains are cultured in MRS for 24 hours at 37° C. in anaerobic condition. The cells are recovered by centrifugation at 10,000 rpm for 10 min then resuspended in a PBS buffer, this step is repeated a second time to wash the bacterial cells. A bacterial suspension is finally prepared with the DMEM medium without addition at a concentration of 10⁷ CFU/mL.

The HT-29 MTX cells are seeded in a 24 well plate at a concentration of 40,000 cells per well in a volume of 500 μL of complete DMEM. The Caco-2 cells are seeded in a 96 well plate at a concentration of 8,000 cells per well in a volume of 200 μL of complete DMEM medium. For another experiment, the Caco-2 cells are seeded on inserts placed in a 12 well plate (polyester membrane, 0.4 μm, Costar®, Corning, New-York, United States) on the apical side in a volume of 500 μL of complete DMEM medium, a volume of 1,500 μL of this same medium is added on the basal side of the insert. All cell cultures are cultured for 15 days with a renewal of the medium every 2 days during the second week of culture. The intestinal cells are washed twice with the PBS buffer before the contact with the bacterial strains. A volume of 150 μL of bacterial suspension is added into each well for a culture in a 96 well plate, and a volume of 300 μL is meanwhile added for a contact in a 24 well plate or in inserts on a 12 well plate. In all cases, contact takes place for 24 h at 37° C., 5% of CO₂. The used negative control is a PBS buffer diluted in a complete DMEM medium.

### Measurement of the Total Calcium Transport

The total calcium transport is defined as being the portion of calcium passing from the apical pole to the basal pole by crossing the epithelial barrier. This transport is assessed by the variation of the calcium concentration at the basal pole of the cell barrier over time. The Caco-2 cells cultured in inserts in a 12 well plate are used for this experiment. At the beginning of the contact with the bacterial strains (t=0 h), 10 μL of a 250 mM calcium chloride (CaCl2)) solution are added on the apical side of the insert. During the contact, samples of 100 μL are taken on the basal side of the insert at 30 min, 7 and 24 h of contact. The samples are stored at −20° C. until analysis. At the time of the different samplings, a measurement of the transepithelial electrical resistance (TEER) is carried out to check the integrity of the Caco-2 cell barrier. This resistance is measured by a MilliCell Electrical Resistance System voltmeter/ohmmeter (Merck Millipore, Burlington, United States). An empty insert without cells is used as a blank to obtain the resistance of the cell barrier. The resistance (in Ω) measured for each well is then multiplied by the area of the insert (equal to 1.12 cm² for the used insert) to give a resistance in Ω·cm⁻². The results are expressed as a percentage of the value obtained at 30 min of contact (set at 100%) for each well. In order to measure the total transport of calcium, an assay of the calcium concentration is carried out in the samples taken at the basal pole of the membrane of Caco-2 cells during contact. The determination of the calcium concentration is carried out using the calcium colorimetric assay kit (Sigma-Aldrich, St Louis, United States). This kit is based on the colorimetric reaction between calcium ions and ortho cresolphthalein forming a colored complex which can be detected at 575 nm. The assay is carried out using 25 μL of samples diluted (1/2) in MiIIi-Q® H₂O, by following the kit protocol. The measurement of the absorbance of the samples at 575 nm is carried out by a Xenius XC spectrofluorimeter (Safas Monaco, Monaco, France). The calcium concentration in the samples is determined by means of a standard range of CaCl2), it is then expressed as a function of the ratio of the concentration at time t to the concentration at time t=30 min of contact.

The results show that the TEER increases during the incubation time with the strains as well as in the control condition (PBS buffer). Thus, for the VFH049 strain, the TEER changes from 100% at t=0 to 245% after 24 hours in a quasi-linear manner. For the VF50b strain, the TEER changes from 100% at t=0 to 231% after 24 hours in a quasi-linear manner. Moreover, it is observed that the strains of the invention do not allow significantly modulating the absorption after 7 hours of contact relative to the PBS buffer. However, after 24 h of incubation, the calcium concentration decreases in the basal compartment for the control condition, while a significant increase in the amount of calcium is observed for the VFH049 and VF50b strains. Thus, for the VFH049 strain, the concentration ratio at t=2 h/concentration at t=0 reaches the value of 1.08 while for the VF50b strain, the same ratio is 1.05.

### Study of the Expression of Different Genes Involved in the Metabolism of Calcium and Vitamin D

In this part, the changes in the expression of several genes after 24 h contact between the bacterial strains and the HT-29 MTX cells were studied. The contact took place in a 24 well plate as previously described. After contact, the HT-29 MTX cells are rinsed twice with the PBS buffer. The RNA extraction is then carried out using the TRIzol™ reagent (Sigma-Aldrich, St Louis, United States). The RNA samples were first treated with DNase to remove the possible DNA fragments co-extracted and/or remaining after extraction. An 8 μL volume containing 1000 ng of RNA is mixed with 1 μL of DNase, and 1 μL of DNase buffer (ThermoScientific, Waltham, United States). The reaction takes place at 37° C. for 30 min in a Mastercycler gradient thermocycler (Eppendorf, Hamburg, Germany), it is stopped by adding 1 μL of 50 mM EDTA solution (ThermoScientific, Waltham, United States) followed by an incubation at 65° C. for 10 min. Subsequently, the samples were retrotranscribed into cDNA using the RevertAid H minus first strand cDNA synthesis kit (ThermoScientific, Waltham, United States), according to the provided instructions. For the qPCR reaction, the cDNA samples are diluted (1/16) in H₂O. For a 2 L volume of sample, 18 μL of qPCR mixture is added containing 10 μL of Power SYBR® Green PCR Master Mix (2×) (Applied Biosystems, Life Technologies, Foster City, United States), 0.6 μL of each primer (10 μM) and 6.8 μL of H₂O. the fluorescence is followed during the reaction in a CFX Connect Real Time Detection System thermocycler (Bio-Rad, Hercules, United States). After a denaturation step at 95° C. for 10 min, 40 PCR cycles are carried out successively, one cycle comprises a denaturation at 95° C. for 15 sec, a hybridization from 58 to 61° C. depending on the pair of primers used for 30 sec and an elongation at 72° C. for 30 sec. The realization of a melting curve ends the reaction. In this part, 4 genes are studied, this is a gene coding for claudin 2 (cId-2), involved in the tight junctions and acting as a channel for the passage of calcium. The gene encoding the vitamin D receptor (vdr) and finally the gene encoding the calcium transporter (trpv6). The expression of these genes is normalized relative to that of the gene encoding peptidylprolyl isomerase A (ppiA) taken as a reference. The pairs of primers used for the studied genes, as well as their hybridization temperatures, are shown in the table below:

TABLE 14

| | | Size of the | |
|---|---|---|---|
| Gene¤ | Primers (5′-3′)¤ | amplicon (pb)¤ | Tm(° C.)¤ |
| vdr¤ | sens: GCCACCTGCTCTATGCCAAG | 171¤ | 61¤ |
| | (SEQ ID NO 204)¶ | | |
| | Anti-sens: CAGGCTGTCCTAGTCAGGAGAT | | |
| | (SEQ ID NO 205)¤ | | |
| trpv5¤ | sens: TGATGCGGCTCATCAGTGCCAGC | 251¤ | 58¤ |
| | (SEQ ID NO 206)¶ | | |
| | anti-sens: GTAGAAGTGGCCTAGCTCCTCG | | |
| | (SEQ ID NO 207)¤ | | |
| cId-2¤ | sens: TGGCCTCTCTTGGCCTCCAACTTGT | 638¤ | 60¤ |
| | (SEQ ID NO 208)¶ | | |
| | anti-sens: TTGACCAGGCCTTGGAGAGCTC | | |
| | (SEQ ID NO 209)¤ | | |
| ppia¤ | sens: TGCTGACTGTGGACAACTCG | 136¤ | 60¤ |
| | (SEQ ID NO 210)¶ | | |
| | anti-sens: TGCAGCGAGAGCACAAAGAT | | |
| | (SEQ ID NO 211)¤ | | |

The results are represented in FIG. 10. The expression of the gene encoding the vitamin D receptor (vdr) is significantly increased in the presence of the VF50b strain; it is increased by 1.8 times relative to the PBS buffer. With regard to the VFH049 strain, it causes an increase in the expression of the gene encoding the calcium transporter (trpv6) by at least 3 times greater than the control. The VF50b strain is the only strain causing a 2.8 times greater expression increase of the cId-2 gene relative to the PBS buffer.

### Fermentation Ability of the VF50b Strain

The growth and acidification properties of the VF50b strain are greater than those of other strains of the same species, which is indeed a particularly interesting strain for the artisanal or industrial production of fermented dairy products (use as a fermentation starter).

Table 15 below compares the properties of the VF50b strain with those of other strains of the same species (tests carried out with cow's milk):

TABLE 15

| | Final concentration in fermentation | Acidification- |
|---|---|---|
| [symbol] | product (OD₆₀₀ [symbol] | (pH [symbol] |
| [symbol] | 0.97 [symbol] | 4.5 [symbol] |
| Strain [symbol] | 0.47 [symbol] | 4.6 [symbol] |
| Strain [symbol] | 0.82 [symbol] | 4.6 [symbol] |

There is a greater growth (higher optical density) and a more efficient acidification (lower pH).

The VF50b strain can be used for manufacturing yogurt, for example.

It is within the reach of the person skilled in the art to consider the study of reverse peptides and dextrorotatory peptides corresponding to the sequences of the peptides of the invention in order to determine whether these peptides have an effect on ACE and/or calcium absorption.

1-30. (canceled)

31. A method of fermentation comprising:
a) contacting a composition comprising a protein substrate with a starter culture under conditions to allow anaerobic fermentation of the protein substrate,
wherein the starter culture comprises at least one of the following strains:
i) Lactobacillus helveticus VF45A strain filed with the CNCM under the order number CNCM-I-5300,
ii) Lactobacillus helveticus VFH049 strain filed with the CNCM under the order number CNCM-I-5403, and
iii) Lactobacillus delbrueckii ssp. bulgaricus VF50b strain filed with the CNCM under the order number CNCM-I-5316,
wherein the protein substrate is not yak's milk or mare's milk.

32. A method of claim 31, wherein the protein substrate comprises cow's milk, goat's milk, sheep's milk or mixtures thereof.

33. A method of claim 31, wherein the protein substrate comprises a plant protein originating from one or more of a cereal, pea, fungus or nut, or mixtures thereof.

34. A method of claim 31, further comprising:
b) isolating a fermentate, and
c) subjecting the fermentate to ultrafiltration at a cut-off threshold of 10 kDa to obtain a filtrate.

35. A composition comprising the fermented protein substrate produced by the method of claim 31.

36. A composition comprising the isolated fermentate produced by the method of claim 34.

37. A composition comprising the filtrate produced by the method of claim 34.

38. A composition comprising at least one microorganism selected from the group consisting of
i) Lactobacillus helveticus VF45A strain filed with the CNCM under the order number CNCM-I-5300,
ii) Lactobacillus helveticus VFH049 strain filed with the CNCM under the order number CNCM-I-5403, and
iii) Lactobacillus delbrueckii ssp. bulgaricus VF50b strain filed with the CNCM under the order number CNCM-I-5316.

39. The composition of claim 38 further comprising vitamin D.

40. The composition of claim 38, wherein the microorganism is microencapsulated.

41. A method of treating a disease or disorder comprising administering to a subject in need thereof a therapeutically effective amount of the composition of claim 35,
wherein the disease or disorder is selected from hypocalcemia, a pathology caused by a deficiency in calcium absorption, arterial hypertension, metabolic syndrome, osteoporosis, and the diseases caused by an abnormality in the regulation of calcemia.

42. A method of treating a disease or disorder comprising administering to a subject in need thereof a therapeutically effective amount of the composition of claim 36, wherein the disease or disorder is selected from hypocalcemia, a pathology caused by a deficiency in calcium absorption, arterial hypertension, metabolic syndrome, osteoporosis, and the diseases caused by an abnormality in the regulation of calcemia.

43. A method of treating a disease or disorder comprising administering to a subject in need thereof a therapeutically effective amount of the composition of claim 37, wherein the disease or disorder is selected from hypocalcemia, a pathology caused by a deficiency in calcium absorption, arterial hypertension, metabolic syndrome, osteoporosis, and the diseases caused by an abnormality in the regulation of calcemia.

44. A method of treating a disease or disorder comprising administering to a subject in need thereof a therapeutically effective amount of the composition of claim 38, wherein the disease or disorder is selected from hypocalcemia, a pathology caused by a deficiency in calcium absorption, arterial hypertension, metabolic syndrome, osteoporosis, and the diseases caused by an abnormality in the regulation of calcemia.

45. A food product or food supplement comprising the composition of claim 35.

46. A food product or food supplement comprising the composition of claim 36.

47. A food product or food supplement comprising the composition of claim 37.

48. A food product or food supplement comprising the composition of claim 39.

49. A food product or food supplement comprising the composition of claim 40.

50. A composition comprising
a) one or more polypeptides selected from the group consisting of polypeptides comprising an amino acid sequence having at least 95% identity to SEQ ID NO: 1-42 or 43;
b) one or more polypeptides selected from the group consisting of polypeptides comprising an amino acid sequence having at least 95% identity to SEQ ID NO: 44-85 or 86;
c) one or more polypeptides selected from the group consisting of polypeptides comprising an amino acid sequence having at least 95% identity to SEQ ID NO: 87-198 or 199.

51. A method of treating a disease or disorder comprising administering to a subject in need thereof a therapeutically effective amount of the composition of claim 50, wherein the disease or disorder is selected from hypocalcemia, a pathology caused by a deficiency in calcium absorption, arterial hypertension, metabolic syndrome, osteoporosis, and the diseases caused by an abnormality in the regulation of calcemia.

52. A food product or food supplement comprising the composition of claim 50.
